(19) United States
(12) Patent Application Publication
(10) Pub. No.: US 2002/0012967 A1
(43) Pub. Date: Jan. 31, 2002

(54) Insulin homolog polypeptide zins4

(76) Inventors: James L. Holloway, Seattle, WA (US); Si Lok, Seattle, WA (US); Stephen R. Jaspers, Edmonds, WA (US)

Correspondence Address: Susan E. Lingenfelter, ZymoGenetics, Inc., 1201 Eastlake Avenue East, Seattle, WA, 98102, US

(21) Appl. No.: 09/781,077

(22) Filed: Feb. 9, 2001

Related U.S. Application Data
(60) Provisional application No. 60/188,544, filed on Mar. 10, 2000.

(51) Int. Cl. C12P 21/02; C07H 21/04; C07K 14/575; C12N 5/06

(52) U.S. Cl. 435/69.4; 435/325; 530/399; 536/23.5

(57) ABSTRACT

The present invention relates to polynucleotide and polypeptide molecules for zins4, a novel polypeptide having homology to the relaxins. The polynucleotides encoding zins4, may, for example, be used to identify a region of the genome associated with human disease states. The present invention also includes methods for producing the protein, uses thereof and antibodies thereto.

### REFERENCE TO RELATED APPLICATIONS

This application is related to Provisional Application 60/188,544, filed on Mar. 10, 2000. Under 35 U.S.C. § 119(e)(1), this application claims benefit of said Provisional Application.

### BACKGROUND OF THE INVENTION

Proliferation and differentiation of cells within multicellular organisms is controlled by hormones and polypeptide growth factors. These diffusable molecules allow cells to communicate with each other and act in concert to form organs, and to repair and regenerate damaged tissue. Examples of hormones and growth factors include the steroid hormones (e.g., estrogen, testosterone), parathyroid hormone, follicle stimulating hormone, the interleukins, platelet derived growth factor (PDGF), epidermal growth factor (EGF), granulocyte-macrophage colony stimulating factor (GM-CSF), erythropoietin (EPO), insulin and calcitonin.

Hormones and growth factors influence cellular metabolism by binding to receptors. Receptors may be integral membrane proteins that are linked to signaling pathways within the cell, such as second messenger systems. Other classes of receptors are soluble molecules, such as certain transcription factors.

Insulin belongs to a group of protein/polypeptide hormones. Insulin increases the rate of synthesis of glycogen, fatty acids, and proteins and stimulates glycolysis. It also promotes the transport of glucose, some other sugars, and amino acids into muscle and fat cells. The mature form of insulin consists of a 30 amino acid residue B chain, that is at the N-terminus of the propeptide form, and a 21 amino acid residue A chain, that is C-terminal. Proinsulin also contains a connecting peptide, C-peptide, between the B chain and A chain that is cleaved out to form mature insulin.

The B chain and A chain are covalently joined by two disulfide bonds. The B-chain, C-peptide, A-chain motif is found in several other proteins including, relaxin (U.S. Pat. No. 4,835,251), insulin-like growth factors (IGF) I and II (Bang and Hall, In “Insulin-like Growth Factors”, P. N. Schofield (eds.), 151-177, Oxford Univ. Press, Oxford), Leydig factor (Bullesbach et al., J. Biol. Chem. 270:16011-15, 1995), Leydig insulin-like peptide (LEY I-L, Burkhardt et al., Hum. Genet. 94:91-4, 1994), early placenta insulin-like factor (EPIL or INSI4; Chassin et al., Genomics 29:465-70, 1995), INSL5 (zins3, Conklin et al., Genomics 60:50-60, 1999), and INSL6 (zins2, Lok et al., WIPO Publication WO98/05785). Unlike the other members of the insulin superfamily, IGF I and IGF II have D and E domains that are cleaved post-translationally. Cysteines that are involved in disulfide bonds are conserved in all the members of the family and play a role in the tertiary structure of the molecules.

Growth factors have had an enormous impact on our understanding of and ability to treat metabolic and cellular disorders, discovery of new factors is important. These and other aspects of the invention will become evident upon reference to the following detailed description of the invention and attached drawings.

### SUMMARY OF THE INVENTION

Within one aspect the invention provides an isolated polypeptide comprising amino acid residues 26-52 of SEQ ID NO:2. Within one embodiment the polypeptide further comprises amino acid residues 119-142 of SEQ ID NO:2. Within a related embodiment the said polypeptide further comprises amino acid residues 55-114 of SEQ ID NO:2. Within another embodiment the polypeptide comprises the amino acid residues 26-142 of SEQ ID NO:2. Within another embodiment polypeptide comprises the amino acid residues 1-142 of SEQ ID NO:2.

The invention also provides an isolated polypeptide having an amino acid sequence that is at least 80% identical to the amino acid sequence of SEQ ID NO:2, wherein said polypeptide specifically binds with an antibody to which a polypeptide having the amino acid sequence of SEQ ID NO:2 specifically binds. Within one embodiment the sequence that is at least 90% identical to the amino acid sequence of SEQ ID NO:2. Within another embodiment the isolated polypeptide comprises the sequences of SEQ ID NO:3, SEQ ID NO:4, and SEQ ID NO:5. Within yet another embodiment any difference between said amino acid sequence and said corresponding amino acid sequence of SEQ ID NO:2 is due to one or more conservative amino acid substitutions. Within a related embodiment the amino acid percent identity is determined using a FASTA program with ktup=l, gap opening penalty=10, gap extension penalty=1, and substitution matrix=blosum62, with other parameters set as default.

Also provided is an isolated polypeptide comprising the amino acid sequence of SEQ ID NO:2. Additionally, the invention provides an isolated polypeptide selected from the group consisting of: a) a polypeptide consisting of the sequence of amino acid residues from residue 26 to residue 52 of SEQ ID NO:2;

b) a polypeptide consisting of the sequence of amino acid residues from residue 26 to residue 53 of SEQ ID NO:2; c) a polypeptide consisting of the sequence of amino acid residues from residue 26 to residue 54 of SEQ ID NO:2; d) a polypeptide consisting of the sequence of amino acid residues from residue 55 to residue 114 of SEQ ID NO:2; e) a polypeptide consisting of the sequence of amino acid residues from residue 55 to residue 115 of SEQ ID NO:2; f) a polypeptide consisting of the sequence of amino acid residues from residue 55 to residue 116 of SEQ ID NO:2; g) a polypeptide consisting of the sequence of amino acid residues from residue 55 to residue 117 of SEQ ID NO:2; h) a polypeptide consisting of the sequence of amino acid residues from residue 55 to residue 118 of SEQ ID NO:2; i) a polypeptide consisting of the sequence of ano acid residues from residue 119 to residue 142 of SEQ ID NO:2; j) a polypeptide consisting of the sequence of amino acid residues from residue 26 to residue 114 of SEQ ID NO:2;

k) a polypeptide consisting of the sequence of amino acid residues from residue 26 to residue 118 of SEQ ID NO:2; 1) a polypeptide consisting of the sequence of amino acid residues from residue 55 to residue 142 of SEQ ID NO:2; m) a polypeptide consisting of the sequence of amino acid residues from residue 1 to residue 25 of SEQ ID NO:2;

n) a polypeptide consisting of the sequence of amino acid residues from residue 1 to residue 52 of SEQ ID NO:2; o) a polypeptide consisting of the sequence of amino acid residues from residue 26 to residue 54 of SEQ ID NO:2; and p) a polypeptide consisting of the sequence of amino acid residues from residue 1 to residue 118 of SEQ ID NO:2.

The invention also provides an isolated protein having: a B chain comprising amino acid residue 26 to amino acid residue 52 of SEQ ID NO:2; and an A chain comprising amino acid residue 119 to amino acid residue 142 of SEQ ID NO:2; wherein the B chain and A chain are joined by inter- and intra-chain disulfide bonds.

Also provided is an isolated polypeptide as described above, further comprising an affinity tag or binding domain.

Within another aspect the invention provides an isolated polynucleotide molecule that encodes a polypeptide as described above. The invention also provides an isolated polynucleotide molecule of SEQ ID NO:1. Additionally the invention provides an isolated polynucleotide selected from the group consisting of: a) a polynucleotide molecule consisting of nucleotides 74-156 of SEQ ID NO:1; b) a polynucleotide consisting of nucleotides 74-159 of SEQ ID NO:l; c) a polynucleotide consisting of nucleotides 74-162 of SEQ ID NO:1; d) a polynucleotide consisting of nucleotides 163-342 of SEQ ID NO:1; e) a polynucleotide consisting of nucleotides 163-345 of SEQ ID NO:1; f) a polynucleotide consisting of nucleotides 163-348 of SEQ ID NO:1; g) a polynucleotide consisting of nucleotides 163-351 of SEQ ID NO:1;

h) a polynucleotide consisting of nucleotides 163-354 of SEQ ID NO:1; i) a polynucleotide consisting of nucleotides 355426 of SEQ ID NO:1; j) a polynucleotide consisting of nucleotides 1-73 of SEQ ID NO:1; k) a polynucleotide consisting of nucleotides 1-162 of SEQ ID NO:1; 1) a polynucleotide consisting of nucleotides 1-342 of SEQ ID NO:1; m) a polynucleotide consisting of nucleotides 74-342 of SEQ ID NO: 1; n) a polynucleotide consisting of nucleotides 74-345 of SEQ ID NO:1; o) a polynucleotide consisting of nucleotides 74-348 of SEQ ID NO:1; p) a polynucleotide consisting of nucleotides 74-351 of SEQ ID NO:1; and q) a polynucleotide consisting of nucleotides 74-354 of SEQ ID NO:1.

The invention also provides an expression vector comprising the following operably linked elements: a transcription promoter; a DNA molecule that encodes a polypeptide as described above; and a transcription terminator.

Additionally the invention provides cultured cell into which has been introduced an expression vector comprising the following operably linked elements: a transcription promoter; a polynucleotide molecule that encodes a polypeptide according to claim 1; and a transcription terminator, wherein said cultured cell expresses said polypeptide encoded by said polynucleotide segment. Within one embodiment the cell further comprises a second expression vector comprising the following operably linked elements: a transcriptional promoter; a DNA sequence encoding a prohormone convertase; and a transcriptional terminator. Within yet another embodiment the prohormone convertase is selected from the group consisting of prohormone convertase ⅓, prohormone convertase 2, prohormone convertase 4, PACE, PACE4, furin, and kex2.

The invention also provides a method of producing a protein comprising: culturing a cell into which has been introduced an expression vector comprising the following operably linked elements: a transcription promoter; a polynucleotide molecule that encodes a polypeptide as described above; and a transcription terminator; whereby said cell expresses said polypeptide encoded by said polynucleotide segment; and recovering said expressed protein.

The invention also provides a fusion protein, comprising the polypeptide as described above.

Within another aspect the invention provides an antibody or antibody fragment that specifically binds to a polypeptide as described above. Within one embodiment the antibody is selected from the group consisting of: a) polyclonal antibody; b) murine monoclonal antibody; c) humanized antibody derived from b); and d) human monoclonal antibody. Within another embodiment the antibody fragment is selected from the group consisting of F(ab′), F(ab), Fab′, Fab, Fv, scFv, and minimal recognition unit. The invention also provides an anti-idiotype antibody that specifically binds to an antibody as described above.

The invention also provides a polypeptide as described above, in combination with a pharmaceutically acceptable vehicle.

The invention further provides a method for producing, from a genomic DNA source, a contiguous polypeptide encoding polynucleotide sequence free from introns comprising the steps of: a) amplifying exon segments using exon specific primer pairs having a type II-S restriction endonuclease site near the primer 5′ terminus; b) digest the amplified exon segments with the type II-S restriction endonuclease to produce cohesive overhangs; and c) ligate digested exon segments. Within one embodiment the genomic DNA source is purified genomic DNA, a genomic clone, a BAC, a PAC, genomic library, or chromosomal DNA clone.

### DETAILED DESCRIPTION OF THE INVENTION

Prior to setting forth the invention in detail, it may be helpful to the understanding thereof to define the following terms:

The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), Glu-Glu affinity tag (Grussenmeyer et al., Proc. Natl. Acad. Sci. USA 82:7952-4, 1985), substance P, Flag™ peptide (Hopp et al., Biotechnology 6:1204-10, 1988), streptavidin binding peptide, or other antigenic epitope or binding domain. See, in general, Ford et al., Protein Expression and Purification 2: 95-107, 1991. DNAs encoding affinity tags are available from commercial suppliers (e.g., Pharmacia Biotech, Piscataway, N.J.).

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The terms “amino-terminal” (N-terminal) and “carboxyl-terminal” (C-terminal) are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “complement/anti-complement pair” denotes non-identical moieties that form a non-covalently associated, stable pair under appropriate conditions. For instance, biotin and avidin (or streptavidin) are prototypical members of a complement/anti-complement pair. Other exemplary complementlanti-complement pairs include receptor/ligand pairs, antibody/antigen (or hapten or epitope) pairs, sense/antisense polynucleotide pairs, and the like. Where subsequent dissociation of the complement/anti-complement pair is desirable, the complement/anti-complement pair preferably has a binding affinity of <10⁹ M⁻¹.

The term “complements of a polynucleotide molecule” denotes a polynucleotide molecule having a complementary base sequence and reverse orientation as compared to a reference sequence. For example, the sequence 5′ ATGCACGGG 3′ is complementary to 5′ CCCGTGCAT 3′.

The term “contig” denotes a polynucleotide that has a contiguous stretch of identical or complementary sequence to another polynucleotide. Contiguous sequences are said to “overlap” a given stretch of polynucleotide sequence either in their entirety or along a partial stretch of the polynucleotide. For example, representative contigs to the polynucleotide sequence 5′-ATGGCTTAGCTT-3′ are 5′-TAGCTTgagtct-3′ and 3′-gtcgacTACCGA-5′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

The term “operably linked”, when referring to DNA segments, indicates that the segments are arranged so that they function in concert for their intended purposes, e.g., transcription initiates in the promoter and proceeds through the coding segment to the terminator.

The term “ortholog” denotes a polypeptide or protein obtained from one species that is the functional counterpart of a polypeptide or protein from a different species. Sequence differences among orthologs are the result of speciation. “Paralogs” are distinct but structurally related proteins made by an organism. Paralogs are believed to arise through gene duplication. For example, α-globin, β-globin, and myoglobin are paralogs of each other.

A “polynucleotide” is a single- or double-stranded polymer of deoxyribonucleotide or ribonucleotide bases read from the 5′ to the 3′ end.

Polynucleotides include RNA and DNA, and may be isolated from natural sources, synthesized in vitro, or prepared from a combination of natural and synthetic molecules.

Sizes of polynucleotides are expressed as base pairs (abbreviated “bp”), nucleotides (“nt”), or kilobases (“kb”). Where the context allows, the latter two termns may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

The term “splice variant” is used herein to denote alternative forms of RNA transcribed from a gene. Splice variation arises naturally through use of alternative splicing sites within a transcribed RNA molecule, or less commonly between separately transcribed RNA molecules, and may result in several mRNAs transcribed from the same gene. Splice variants may encode polypeptides having altered amino acid sequence. The term splice variant is also used herein to denote a protein encoded by a splice variant of an MRNA transcribed from a gene.

Molecular weights and lengths of polymers determined by imprecise analytical methods (e.g., gel electrophoresis) will be understood to be approximate values. When such a value is expressed as “about” X or “approximately” X, the stated value of X will be understood to be accurate to ±10%.

All references cited herein are incorporated by reference in their entirety.

The present invention is based in part upon the discovery of a novel DNA sequence that encodes a novel polypeptide having homology to the relaxin family. The polypeptide has been designated zins4.

A genomic DNA sequence was discovered and exons were predicted to code for a secreted full-length protein. The nucleotide sequence encoding a full-length zins4 polypeptide is described in SEQ ID NO:1, and its deduced amino acid sequence is described in SEQ ID NO:2. The mature polypeptide has homology to the relaxin family. The zins4 polypeptide of SEQ ID NO:2 shares 28% identity over a 117 amino acid residue overlap with spiny dogfish relaxin (RELX_SQUAC, Bullesbach et al., Eur. J. Biochem. 161:335-41, 1986); and 20.6% identity over a 194 amino acid residue overlap with human prorelaxin H2 precursor (REL2_Human, Hudson et al., EMBO J. 3:2333-39, 1984). Percent identity was determined using the Smith-Waterman algorithm (Smith and Waterman, J. Mol. BioL 147:195-7, 1981; and Pearson, Genomics 11:635-50, 1991).

Within the relaxin family the cysteine motif is highly conserved in the B and A chains, where the B chain motif can be represented as LCGX{10}C, where X{ } is the number of amino acid residues. X is any amino acid residue except cysteine. The A chain motif is CCX{3}CX{8}C (SEQ ID NO:4), where X{ } is the number of any amino acid residues. X is any amino acid residue except cysteine. The B chain also contains a conserved motif RXXXR (SEQ ID NO:5). X represents any amino acid residue except cysteine. The two arginine residues are necessary for relaxin binding to cells and tissues. If either arginine is changed synthetically, there is no competition for relaxin binding.

The DNA sequence encoding the zins4 polypeptide revealed that the predicted amino acid sequence contained the B chain-C peptide-A chain motif found in the relaxins and insulin. Preprorelaxin and preproinsulin both have a signal sequence, followed by the B chain, C peptide, and then the A chain. The mature relaxins and insulin have the signal peptide and C peptide removed, with the B and A chains joined by both inter- and intra-chain disulfide bonds (James et al., Nature 267:544-546, 1977). Sequence analysis indicates that the human polypeptide sequence (SEQ ID NO:2) is structurally equivalent to other members of the family.

Processing of the protein involves cleavage at the C-terminus of the signal peptide, and, based on predicted structural homology with other mature members of the insulin family, a cleavage at the C-terminus of the B chain and at the N-terminus of the A chain, resulting in removal of the C-peptide. Analysis of the zins4 polypeptide of SEQ ID NO:2 with other known members of the insulin family suggests a signal peptide cleavage site in the region of amino acid residue 25 (Ala) of SEQ ID NO:2.

Cleavage at the C-terminus of the B chain is predicted to be at the C-terminal of amino acid residue 53 (Arg) or residue 54 (Arg) followed by cleavage of the Arg residues by carboxypeptidase to leave amino acid residue 52 (Trp) as the C-terminal amino acid residue. Cleavage sites resulting in the N-terminus of the A chain are suggested in the region of amino acid residue 115 (Arg) to 118 (Arg). Cleavage is predicted to be after the C-terminus of amino acid residue 118 (Arg) leaving amino acid residue 119 (Asp) as the N-terminal amino acid residue of the A chain. The C-terminal amino acid is residue 142 (Cys). The cleavage site at the junction of the C-peptide and A chain is highly conserved, occurring after Arg-X-X-Arg (wherein X is any amino acid residue), Arg-Arg or Lys-Arg; however, the cleavage sites at the junction of the signal sequence and B chain, and at the junction of the B chain and C-peptide, do not maintain a similarly high degree of conservation within the insulin family. Based on the predicted sites, the amino acid sequence of mature zins4 includes a B chain having the sequence of SEQ ID NO:2 from amino acid residue 26 (Arg) to amino acid residue 52 (Trp), and an A chain having the sequence of SEQ ID NO:2 from amino acid residue 119 (Asp) to amino acid residue 142 (Cys).

The enzymology of proinsulin conversion suggests that prohormone convertase ⅓ (PC⅓), or prohormone convertase 2 (PC2), cleave zins4 primarily at the B chain-C-peptide junction. Following cleavage at this site, it is predicted that a carboxypeptidase will remove the resulting C-terminal arginine residues and leave amino acid residue 30 (Trp) of SEQ ID NO:2 as the C-terminal residue of the B-chain in the mature hormone. Members of the furin family of convertases, furin, PACE, PACE4, and prohormone convertase 4 (PC4) cleave preferentially at the C-peptide-A-chain junction. Also useful is the yeast dibasic-specific endoprotease Kex2. Co-expression of zins4 with an appropriate member of the furin family of prohormone convertases will yield a mature hormone with amino acid residue 119 (Asp) of SEQ ID NO:2 as the N-terminal residue of the A-chain. This is consistent with the relaxin family since mouse, dog, tammar, shark, and skate relaxins and murine INSL5 all have acidic amino acids at the N-terminus of the A chain. Within the relaxin proteins there is a highly conserved region in the B chain represented by the motif RXXXR (amino acid residues 37-41 of SEQ ID NO:2) and the motif LCGX{10}C (amino acid residues 34-47 of SEQ ID NO:2), where X{ } is the number of any amino acid residues, and X is any amino acid residue except cysteine. The A chain contains a conserved motif CCX{3}CX{8}C (amino acid residues 128-142 of SEQ ID NO:2), where X{ } is the number of any amino acid residues, and X is any amino acid residue except cysteine.

The C peptide comprises amino acid residue 55 (Ser) to amino acid residue 118 (Arg) of SEQ ID NO:2 The C peptide from insulin, when administered to diabetic rats, decreased or prevented diabetes-induced damage to neurons or blood vessels (Ido et al., Science 277:563-66, 1997).

Chromosomal localization of zins4 has been made to chromosome 19pl3.11. As used herein zins4 designates nucleotide sequence or related matters and zins4 designates polypeptide sequences.

The present invention also provides polynucleotide molecules, including DNA and RNA molecules, that encode the zins4 polypeptides disclosed herein. Those skilled in the art will readily recognize that, in view of the degeneracy of the genetic code, considerable sequence variation is possible among these polynucleotide molecules. SEQ ID NO:6 is a degenerate DNA sequence that encompasses all DNAs that encode the zins4 polypeptide of SEQ ID NO:2. Those skilled in the art will recognize that the degenerate sequence of SEQ ID NO:6 also provides all RNA sequences encoding polypeptides of SEQ ID NO:2 by substituting U for T. Thus, zins4 polypeptide-encoding polynucleotides comprising nucleotide 1 to nucleotide 429 of SEQ ID NO:1 and their RNA equivalents are contemplated by the present invention.

Table 1 sets forth the one-letter codes used within SEQ ID NO:6 to denote degenerate nucleotide positions. “Resolutions” are the nucleotides denoted by a code letter. “Complement” indicates the code for the complementary nucleotide(s). For example, the code Y denotes either C or T, and its complement R denotes A or G, A being complementary to T, and G being complementary to C.

TABLE 1

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons used in SEQ ID NO:6, encompassing all possible codons for a given amino acid, are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CPA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATM |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | • | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the amino acid sequence of SEQ ID NO:2. Variant sequences can be readily tested for functionality as described herein.

One of ordinary skill in the art will also appreciate that different species can exhibit “preferential codon usage.” In general, see, Grantham, et al., Nuc. Acids Res. 8:1893-912, 1980; Haas, et al. Curr. BioL 6:315-24, 1996; Wain-Hobson, et al., Gene 13:355-64, 1981; Grosjean and Fiers, Gene 18:199-209, 1982; Holm, Nuc. Acids Res. 14:3075-87, 1986; Ikemura, J. Mol. Biol. 158:573-97, 1982. As used herein, the term “preferential codon usage” or “preferential codons” is a term of art referring to protein translation codons that are most frequently used in cells of a certain species, thus favoring one or a few representatives of the possible codons encoding each amino acid (See Table 2). For example, the amino acid threonine (Thr) may be encoded by ACA, ACC, ACG, or ACT, but in mammalian cells ACC is the most commonly used codon; in other species, for example, insect cells, yeast, viruses or bacteria, different Thr codons may be preferential. Preferential codons for a particular species can be introduced into the polynucleotides of the present invention by a variety of methods known in the art. Introduction of preferential codon sequences into recombinant DNA can, for example, enhance production of the protein by making protein translation more efficient within a particular cell type or species. Therefore, the degenerate codon sequence disclosed in SEQ ID NO:6 serves as a template for optimizing expression of polynucleotides in various cell types and species commonly used in the art and disclosed herein. Sequences containing preferential codons can be tested and optimized for expression in various species, and tested for functionality as disclosed herein.

Within preferred embodiments of the invention the isolated polynucleotides will hybridize to similar sized regions of SEQ ID NO:1, or a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Numerous equations for calculating Tₘ are known in the art, and are specific for DNA, RNA and DNA-RNA hybrids and polynucleotide probe sequences of varying length (see, for example, Sambrook et al., Molecular Cloning: A Laboratory Manual, Second Edition (Cold Spring Harbor Press 1989); Ausubel et al., (eds.), Current Protocols in Molecular Biology (John Wiley and Sons, Inc. 1987); Berger and Kimmel (eds.), Guide to Molecular Cloning Techniques, (Academic Press, Inc. 1987); and Wetmur, Crit. Rev. Biochem. Mol. Biol. 26:227 (1990)). Sequence analysis software such as OLIGO 6.0 (LSR; Long Lake, Minn.) and Primer Premier 4.0 (Premier Biosoft International; Palo Alto, Calif.), as well as sites on the Internet, are available tools for analyzing a given sequence and calculating Tₘ based on user defined criteria. Such programs can also analyze a given sequence under defined conditions and identify suitable probe sequences. Typically, hybridization of longer polynucleotide sequences, >50 base pairs, is performed at temperatures of about 20-25° C. below the calculated Tₘ. For smaller probes, <50 base pairs, hybridization is typically carried out at the Tm or 5-10° C. below. This allows for the maximum rate of hybridization for DNA-DNA and DNA-RNA hybrids. Higher degrees of stringency at lower temperatures can be achieved with the addition of formamide which reduces the Tₘ of the hybrid about 10° C. for each 1% formamide in the buffer solution. Suitable stringent hybridization conditions are equivalent to about a 5 h to overnight incubation at about 42° C. in a solution comprising: about 40-50% formamide, up to about 6×SSC, about 5×Denhardt's solution, zero up to about 10% dextran sulfate, and about 10-20 μg/ml denatured commercially-available carrier DNA. Generally, such stringent conditions include temperatures of 20-70° C. and a hybridization buffer containing up to 6×SSC and 0-50% formamide; hybridization is then followed by washing filters in up to about 2×SSC. For example, a suitable wash stringency is equivalent to 0.1×SSC to 2×SSC, 0.1% SDS, at 55° C. to 65° C. Different degrees of stringency can be used during hybridization and washing to achieve maximum specific binding to the target sequence. Typically, the washes following hybridization are performed at increasing degrees of stringency to remove non-hybridized polynucleotide probes from hybridized complexes. Stringent hybridization and wash conditions depend on the length of the probe, reflected in the Tₘ, hybridization and wash solutions used, and are routinely determined empirically by one of skill in the art.

As previously noted, the isolated polynucleotides of the present invention include DNA and RNA. Methods for preparing DNA and RNA are well known in the art and are discussed herein. Zins4 is representative of an emerging class of genes that were first discovered through interrogation of genomic databases. The absence of information pointing to a source of mRNA template makes it difficult to employ conventional means to isolate a full-length cDNA for these genes.

Additionally, many of these genes are not represented in current EST databases suggesting that they are either expressed at very low levels or by limited cell-types or at restricted times.

Isolation of a cDNA encoding the full-length polypeptide is a necessary first step for producing recombinant protein. While it is possible for some eukaryotic expression system to produce recombinant proteins encoded by introduced genomic sequences, many genes contain multiple introns whose collective length renders the expression unit too large to be efficiently inserted in most plasmid-based expression vectors. Another challenge posed by genomic sequence is the possible presence of repeated elements within introns. These elements may promote plasmid-instability while the expression vector is being propagated through the bacterial host. The presence of intronic sequences within the expression cassette leaves open the possibility for the recipient mammalian host cell to use cryptic splice donor and acceptor sites within the introns. Such alternative splice sites may be natural to the gene or may be artifactural to the expression host cell. The use of these cryptic splice sites by the host cell could lead to the production of a different recombinant polypeptide than intended. The splicing mechanisms of mammalian, yeast or insect cells may also be sufficiently different from one another to preclude accurate or efficient splicing of some mRNAs transcribed by heterologous genes. Finally, the lack of MRNA splicing in bacterial host cells necessitates the removal of all intronic sequence within the expression unit for the production of recombinant protein in such systems. Hence, there is a need for a rapid and efficient method to convert gene sequence to a contiguous polypeptide coding sequence that is free of introns.

The polymerase chain reaction (PCR) provides a convenient method for isolating defined DNA segments through the use of sequence specific pairs of oligonucleotide primers. While the use of PCR and exon-specific primers would enable the isolation of exon gene segments, rejoining the resulting exon segments to produce a contiguous sequence encoding the polypeptide of interest is difficult. To overcome this problem, oligonucleotide primers can be designed that incorporate the polynucleotide sequence of a type II restriction endonuclease cleavage site near the 5′ end of each member of the primer pair. The resulting PCR product derived from these primers would incorporate the restriction endonuclease site at its terminus, which upon digestion with the corresponding type II restriction enzyme, would produce suitable cohesive overhangs to promote efficient ligation in the presence of DNA ligase. This method, however, cannot be employed to ligate exon segments to produce a contiguous polypeptide coding sequence. Exon segments ligated together in this manner would contain a foreign restriction endonuclease recognition sequence between ligated segments thereby introducing one or more added amino acid residues at each ligation junction.

At high DNA and DNA ligase concentrations, it is possible to ligate blunt-ended DNA segments together. The so called blunt-end ligation reaction would enable the ligation of PCR generated exon segments without the need to incorporate restriction endonuclease sites to the primers and thereby the ligated products would be free of foreign sequences. This approach, however, has severe limitations. Since the blunt-end ligation reaction does not make use of defined cohesive ends, the number of combinations and permutations of incorrect ligation products increases exponentially with the number of exons to be ligated, rendering this method impractical for general use.

To overcome these obstacles a new method was devised for generating exon fragments for rapid and efficient ligation to produce a contiguous polypeptide coding sequence that is free of intron or other non-coding polynucleotide sequences. This method makes use of specific primer pairs that are designed to amplify each exon of a gene from either purified genomic DNA template or from genomic DNA inserts cloned in BAC, PAC or the other vectors. Members of each exon-specific primer pair are designed with a suitable type II-S restriction endonuclease site in the appropriate orientation near the 5′ terminus. In contrast to the type II restriction endonuclease, the type II-S restriction enzymes cleave double-stranded DNA and generate complementary cohesive ends at a defined distance and orientation outside an asymmetric DNA recognition site. This property of he type II-S endonucleases, was exploited to design sets of exon-specific PCR primers. Digestion of the resulting amplified exon products produces cohesive overhangs consisting entirely of exon derived sequences. To achieve the ligation of exon segments in the correct order and orientation, exon-primer-pairs were designed such that adjacent exon segments have unique but complementary cohesive overhangs. In this way, ligation of a complex exon mixture is accomplished by cohesive end ligation in the correct orientation and order, without the addition of sequences encoding foreign amino acids as is the case where type-II endonuclease sites were incorporated into the PCR primers.

This method can be used to convert zins4 genomic DNA into a contiguous polypeptide coding sequence (cPCS). The human zcytor-1 gene (Sprecher et al., Biochem. Biophys. Res. Comm. 246:82-90, 1998; Baumgartner et al., U.S. Pat. No. 5,792,850) is located 2 kb from the zins4 locus. Zcytor-1 gene primers were used to isolate a BAC clone containing the gene encoding human zins4. A “Down to the Well” human BAC II DNA library (Genome Systems Inc, St. Louis, Mo.) was screened by PCR according to the manufacturer's instructions using zins4-specific sense and antisense primers ZC9736 (SEQ ID NO:7) and ZC9740 (SEQ ID NO:8). A positive genomic BAC clone (328h12) containing the zins4 gene was identified. DNA from clone 328h12 was prepared for use- as a PCR template using an AutoGen 740 automated DNA extraction system (AutoGen, Framingham, Mass.) in accordance with the manufacturer's instructions.

Zins4 contains two exons. The zins4 exon 1 DNA segment is isolated using sense oligonucleotide primer (SEQ ID NO:9); and antisense oligonucleotide primer (SEQ ID NO:10), respectively. The zins4 exon 2 DNA segment is isolated using sense oligonucleotide primer (SEQ ID NO:11) and antisense oligonucleotide primer (SEQ ID NO:12), respectively. A PCR reaction mix is prepared as follows: to a final volume of 100 μl is added 10 μl 10×native PFU DNA polymerase buffer (Stratagene, La Jolla, Calif.); 1 μl 20 mM dNTP mix; 30 pmoles each of sense and antisense primers; 10 ng of 218hI2 BAC clone DNA template; and 2 units of native PFU DNA polymerase (Stratagene). DNA amplification is carried out as follows: 1 cycle (95° C. for 1 minute), followed by 3 cycle of (95 C for 10 seconds; 45° C. for 10 seconds; 72° C. for 2 minutes) followed by 17 cycles of (95 C for 10 seconds; 60° C. for seconds; 72° C. for 2 minutes), followed by a further 5 minute extension at 72 C. The amplified zins4 exon products are extracted with phenol and chloroform and are precipitated with ethanol. The PCR products are digested with the type-IIS restriction endonuclease, Eco31I (MBI Fermentas, Amherst, N.Y.) according to manufacturer's instruction. The digested PCR product is purified using a Gel Extraction Kit (Qiagen, Chatsworth, Calif.) according to manufacturer's instructions.

Ligation of zins4 exons into EcoR-1 and Xba-1 digested mammalian expression vector pSAM-8 is carried out in a 5 pl ligation reaction consisted of: 1 μl vector (40 ng); 0.5 μl 10×Promega ligase buffer (Promega Life Science, Madison, Wis.), 0.5 μl 10 mM ATP, 1 μl gel purified Eco31I digested zins4 exon 1 (˜10 ng); 1 μl gel purified Eco31I digested zins4 exon 2 (˜10 ng); and 0.5 μl (15 units/1μl ) T4 DNA Ligase (Promega Life Science). The ligation reaction is carried at 6° C. for 2 hrs; 8° C. for 2 hrs; 10° C. for 2 hrs; 12.5° C. for 2 hrs; 15° C. for 2 hrs; followed by an overnight incubation at 10° C. overnight. One μl of the ligation mixture is used to transform MAX efficiency DH10B competent cells (Life Technologies, Rockville, Md.). Transformants are selected by plating onto Ampicillin LB agar plates. The resulting clone carrying the zins4 cPCS is confirmed by DNA sequence analysis.

RNA may also be isolated from a tissue or cell that produces large amounts of zins4 RNA. Such tissues and cells are identified by using the sequence information provide herein in combination with Northern blotting (Thomas, Proc. Natl. Acad. Sci. USA 77:5201, 1980). Total RNA can be prepared using guanidinium isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding zins4 polypeptides are then identified and isolated by, for example, hybridization or PCR. A full-length clone encoding zins4 can be obtained by conventional cloning procedures. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to zins4, receptor fragments, or other specific binding partners.

The polynucleotides of the present invention can also be synthesized using DNA synthesis machines. If chemically synthesized double stranded DNA is required for an application such as the synthesis of a DNA or a DNA fragment, then each complementary strand is made separately, for example via the phosphoramidite method known in the art. The production of short polynucleotides (60 to 80 bp) is technically straightforward and can be accomplished by synthesizing the complementary strands and then annealing them. However, for producing longer polynucleotides (longer than about 300 bp), special strategies are usually employed. For example, synthetic DNAs (double-stranded) are assembled in modular form from single-stranded fragments that are from 20 to 100 nucleotides in length. One method for building a synthetic DNA involves producing a set of overlapping, complementary oligonucleotides. Each internal section of the DNA has complementary 3′ and 5 terminal extensions designed to base pair precisely with an adjacent section. After the DNA is assembled, the process is completed by ligating the nicks along the backbones of the two strands. In addition to the protein coding sequence, synthetic DNAs can be designed with terminal sequences that facilitate insertion into a restriction endonuclease site of a cloning vector. Alternative ways to prepare a full-length DNA are also known in the art. See Glick and Pasternak, Molecular Biotechnology, Principles & Applications of Recombinant DNA, (ASM Press, Washington, D.C. 1994); Itakura et al., Annu. Rev. Biochem. 53: 323-56, 1984 and Climie et al., Proc. Natl. Acad. Sci. USA 87:633-7, 1990.

The present invention further provides counterpart polypeptides and polynucleotides from other species (orthologs). These species include, but are not limited to mammalian, avian, amphibian, reptile, fish, insect and other vertebrate and invertebrate species. Of particular interest are zins4 polypeptides from other mammalian species, including murine, porcine, ovine, bovine, canine, feline, equine, and other primate polypeptides. Orthologs of human zins4 can be cloned using information and compositions provided by the present invention in combination with conventional cloning techniques. For example, a cDNA can be cloned using mRNA obtained from a tissue or cell type that expresses zins4 as disclosed herein. Suitable sources of mRNA can be identified by probing Northern blots with probes designed from the sequences disclosed herein. A library is then prepared from mRNA of a positive tissue or cell line. A zins4-encoding cDNA can then be isolated by a variety of methods, such as by probing with a complete or partial human cDNA or with one or more sets of degenerate probes based on the disclosed sequences. A cDNA can also be cloned using the polymerase chain reaction, or PCR (Mullis, U.S. Pat. No. 4,683,202), using primers designed from the representative human zins4 sequence disclosed herein. Within an additional method, the cDNA library can be used to transform or transfect host cells, and expression of the cDNA of interest can be detected with an antibody to zins4 polypeptide. Similar techniques can also be applied to the isolation of genomic clones.

Those skilled in the art will recognize that the sequence disclosed in SEQ ID NO:1 represents a single allele of human zins4 and that allelic variation and alternative splicing are expected to occur. Allelic variants of this sequence can be cloned by probing cDNA or genomic libraries from different individuals according to standard procedures. Allelic variants of the DNA sequence shown in SEQ ID NO:1, including those containing silent mutations and those in which mutations result in amino acid sequence changes, are within the scope of the present invention, as are proteins which are allelic variants of SEQ ID NO:2. cDNAs generated from alternatively spliced mRNAs, which retain the properties of the zins4 polypeptide are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art.

The present invention also provides isolated zins4 polypeptides that are substantially similar to the polypeptides of SEQ ID NO:2 and their orthologs. The term “substantially similar” is used herein to denote polypeptides having 70%, preferably 75%, more preferably at least 80%, sequence identity to the sequences shown in SEQ ID NO:2 or their orthologs. Such polypeptides will more preferably be at least 90% identical, and most preferably 95% or more identical to SEQ ID NO:2 or its orthologs.) Percent sequence identity is determined by conventional methods. See, for example, Altschul et al., Bull. Math. Bio. 48: 603-16, 1986 and Henikoff and Henikoff, Proc. Natl. Acad. Sci. USA 89:10915-9, 1992. Briefly, two amino acid sequences are aligned to optimize the alignment scores using a gap opening penalty of 10, a gap extension penalty of 1, and the “blosum 62” scoring matrix of Henikoff and Henikoff (supra.) as shown in Table 3 (amino acids are indicated by the standard one-letter codes). The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

TABLE 3

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Those skilled in the art appreciate that there are many established algorithms available to align two amino acid sequences. The “FASTA” similarity search algorithm of Pearson and Lipman is a suitable protein alignment method for examining the level of identity shared by an amino acid sequence disclosed herein and the amino acid sequence of a putative variant zins4. The FASTA algorithm is described by Pearson and Lipman, Proc. Nat'l Acad. Sci. USA 85:2444, 1988, and by Pearson, Meth. Enzymol. 183:63, 1990.

Briefly, FASTA first characterizes sequence similarity by identifying regions shared by the query sequence (e.g., SEQ ID NO:2) and a test sequence that have either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444, 1970; Sellers, SIAM J. Appl. Math. 26:787, 1974), which allows for amino acid insertions and deletions. Preferred parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63, 1990.

FASTA can also be used to determine the sequence identity of nucleic acid molecules using a ratio as disclosed above. For nucleotide sequence comparisons, the ktup value can range between one to six, preferably from three to six, most preferably three, with other parameters set as default.

The BLOSUM62 table (Table 3) is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915, 1992). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed below), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Variant zins4 polypeptides or substantially homologous zins4 polypeptides are characterized as having one or more amino acid substitutions, deletions or additions. These changes are preferably of a minor nature, that is conservative amino acid substitutions (see Table 4) and other substitutions that do not significantly affect the folding or activity of the polypeptide; small deletions, typically of one to about 30 amino acids; and amino- or carboxyl-terminal extensions, such as an amino-terminal methionine residue, a small linker peptide of up to about 20-25 residues, or an affinity tag. The present invention thus includes polypeptides of from about 14 to about 500 amino acid residues that comprise a sequence that is at least 80%, preferably at least 90%, and more preferably 95% or more identical to the corresponding region of SEQ ID NO:2.

TABLE 4
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

The proteins of the present invention can also comprise non-naturally occurring amino acid residues. Non-naturally occurring amino acids include, without limitation, trans-3-methylproline, 2,4-methanoproline, cis-4-hydroxyproline, trans-4-hydroxyproline, N-methylglycine, allo-threonine, methylthreonine, hydroxy-ethylcysteine, hydroxyethylhomocysteine, nitroglutamine, homoglutamine, pipecolic acid, thiazolidine carboxylic acid, dehydroproline, 3- and 4-methylproline, 3,3-di-methylproline, tert-leucine, norvaline, 2-azaphenylalanine, 3-azaphenylalanine, 4-azaphenylalanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated MRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the protein in place of its natural counterpart. See, Koide et al., Biochem. 33:7470-6, 1994. Naturally occurring amino acid residues can be converted to non-naturally occurring species by in vitro chemical modification.

Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for zins4 amino acid residues.

Essential amino acids in the polypeptides of the present invention can be identified according to procedures known in the art, such as site-directed mutagenesis or alanine-scanning mutagenesis (Cunningham and Wells, Science 244: 1081-5, 1989; Bass et al., Proc. Natl. Acad. Sci. USA 88:4498-502, 1991). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699-708, 1996. Sites of ligand-receptor or other biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related polypeptide sequences or proteins.

Determination of amino acid residues that are within regions or domains that are critical to maintaining structural integrity can be determined. Within these regions one can determine specific residues that will be more or less tolerant of change and maintain the overall tertiary structure of the molecule. Methods for analyzing sequence structure include, but are not limited to, alignment of multiple sequences with high amino acid or nucleotide identity and computer analysis using available software (e.g., the Insight II® viewer and homology modeling tools; MSI, San Diego, Calif.), secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules. -Amino acid sequence changes are made in zins4 polypeptides so as to -minimize disruption of higher order structure essential to biological activity. For example, when the zins4 polypeptide comprises one or more conserved structures, changes in amino acid residues will be made so as not to disrupt the structures and other components of the molecule where changes in conformation abate some critical function, for example, binding of the molecule to its binding partners. The effects of amino acid sequence changes can be predicted by, for example, computer modeling as disclosed herein or determined by analysis of crystal structure (see, e.g., Lapthom et al., Nat. Struct. Biol. 2:266-268, 1995). Other techniques that are well known in the art compare folding of a variant protein to a standard molecule (e.g., the native protein).

For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same disulfide bonding pattern as the standard molecule folding would be affected. Another well known and accepted method for measuring folding is circular dichrosism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structural similarities between proteins and polypeptides (Schaanan et al., Science 257:961-964, 1992).

A Hopp/Woods hydrophilicity profile of the zins4 protein sequence as shown in SEQ ID NO:2 can be generated (Hopp et al., Proc. Natl. Acad. Sci.78:3824-3828, 1981; Hopp, J. Immun. Meth. 88:1-18, 1986 and Triquier et al., Protein Engineering 11:153-169, 1998). Those skilled in the art will recognize that hydrophilicity or hydrophobicity will be taken into account when designing modifications in the amino acid sequence of a zins4 polypeptide, so as not to disrupt the overall structural and biological profile. Of particular interest for replacement are hydrophobic residues selected from the group consisting of Val, Leu and lie or the group consisting of Met, Gly, Ser, Ala, Tyr and Trp. For example, residues tolerant of substitution could include these hydrophobic residues as shown in SEQ ID NO:2. Cysteine residues in the cysteine motifs of SEQ ID NO:2, described herein, will be relatively intolerant of substitution.

The identities of essential amino acids can also be inferred from analysis of sequence similarity between insulin/relaxin family members and zins4. Using methods such as “FASTA” analysis described previously, regions of high similarity are identified within a family of proteins and used to analyze amino acid sequence for conserved regions. An alternative approach to identifying a variant zins4 polynucleotide on the basis of structure is to determine whether a nucleic acid molecule encoding a potential variant zins4 polynucleotide can hybridize to a nucleic acid molecule having the nucleotide sequence of SEQ ID NO:1, as discussed above.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl. Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant mutant molecules are tested for biological activity as disclosed below to identify amino acid residues that are critical to the activity of the molecule. See also, Hilton et al., J. Biol. Chem. 271:4699 (1996).

The present invention also includes functional fragments of zins4 polypeptides and nucleic acid molecules encoding such functional fragments. A “functional” zins4 or fragment thereof defined herein is characterized by its proliferative or differentiating activity, by its ability to induce or inhibit specialized cell functions, or by its ability to bind specifically to an anti-zins4 antibody or zins4 receptor (either soluble or immobilized). As previously described herein, zins4 is characterized by several cleavage sites that generate a number of bioactive zins4 peptides. Thus, the present invention further provides fusion proteins encompassing: (a) polypeptide molecules comprising one or more of the of the zins4 peptides described above; and (b) functional fragments comprising one or more of these peptides. The other polypeptide portion of the fusion protein may be contributed by another peptide hormone, such as insulin, glucagon, POMC, growth hormone, neuropeptide hormones, and the like, or by a non-native and/or an unrelated secretory signal peptide that facilitates secretion of the fusion protein.

Routine deletion analyses of nucleic acid molecules can be performed to obtain functional fragments of a nucleic acid molecule that encodes a zins4 polypeptide. As an illustration, DNA molecules having the nucleotide sequence of SEQ ID NO:1 or fragments thereof, can be digested with Bal31 nuclease to obtain a series of nested deletions. These DNA fragments are then inserted into expression vectors in proper reading frame, and the expressed polypeptides are isolated and tested for zins4 activity, or for the ability to bind anti-zins4 antibodies or zins4 receptor. One alternative to exonuclease digestion is to use oligonucleotide-directed mutagenesis to introduce deletions or stop codons to specify production of a desired zins4 fragment.

Alternatively, particular fragments of a zins4 polynucleotide can be synthesized using the polymerase chain reaction.

Standard methods for identifying functional domains are well-known to those of skill in the art. For example, studies on the truncation at either or both termini of interferons have been summarized by Horisberger and Di Marco, Pharmac. Ther. 66:507 (1995). Moreover, standard techniques for functional analysis of proteins are described by, for example, Treuter et al., Molec. Gen. Genet. 240:113 (1993); Content et al., “Expression and preliminary deletion analysis of the 42 kDa 2-5 A synthetase induced by human interferon,” in Biological Interferon Systems, Proceedings of ISIR-TNO Meeting on Interferon Systems, Cantell (ed.), pages 65-72 (Nijhoff 1987); Herschman, “The EGF Receptor,” in Control of Animal Cell Proliferation 1, Boynton et al., (eds.) pages 169-199 (Academic Press 1985); Coumailleau et al., J. Biol. Chem. 270:29270 (1995); Fukunaga et al., J. Biol. Chem. 270:25291 (1995); Yamaguchi et al., Biochem. Pharmacol. 50:1295 (1995); and Meisel et al., Plant Molec. Biol. 30:1 (1996).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Variants of the disclosed zins4 DNA and polypeptide sequences can be generated through DNA shuffling as disclosed by Stemmer, Nature 370:389-91, 1994, Stemmer, Proc. Natl. Acad. Sci. USA 91:10747-51, 1994 and WIPO Publication WO 97/20078. Briefly, variant DNAs are generated by in vitro homologous recombination by random fragmentation of a parent DNA followed by reassembly using PCR, resulting in randomly introduced point mutations. This technique can be modified by using a family of parent DNAs, such as allelic variants or DNAs from different species, to introduce additional variability into the process. Selection or screening for the desired activity, followed by additional iterations of mutagenesis and assay provides for rapid “evolution” of sequences by selecting for desirable mutations while simultaneously selecting against detrimental changes.

Mutagenesis methods as disclosed herein can be combined with high-throughput, automated screening methods to detect activity of cloned, mutagenized polypeptides in host cells. Mutagenized DNA molecules that encode active polypeptides (e.g., secreted and detected by antibodies, binding assays, or measured by a signal transduction type assay) can be recovered from the host cells and rapidly sequenced using modem equipment. These methods allow the rapid determination of the importance of individual amino acid residues in a polypeptide of interest, and can be applied to polypeptides of unknown structure. Using the methods discussed herein, one of ordinary skill in the art can identify and/or prepare a variety of polypeptides that are substantially similar to SEQ ID NO:2 or allelic variants thereof and retain the properties of the wild-type protein. For example, using the methods described above, one could identify a receptor binding domain on zins4; an extracellular ligand-binding domain of a receptor for zins4; other functional or structural domains; affinity tags; or other domains important for protein-protein interactions or signal transduction. Such polypeptides may also include additional polypeptide segments as generally disclosed above.

For any zins4 polypeptide, including variants and fusion proteins, one of ordinary skill in the art can readily generate a fully degenerate polynucleotide sequence encoding that variant using the information set forth in Tables 1 and 2 above.

Zins4 chimeric molecules are also provided by the invention. Zins4 in combination with other members of the insulin/relaxin family can be use to form zwitterhormons, proteins having two unrelated biological functions (Bullesbach et al., Biochem. 35:9754-60, 1996). Zins4 can be combined with insulin, relaxins, insulin-like growth factors, Leydig factors, Leydig insulin-like peptides, early placenta insulin-like factors, INSL5, INSL6, bombyxins, and their analogs, among others. The zins4 polypeptides of the present invention, including full-length polypeptides, the A chain, the B chain, the C peptide as described herein, biologically active fragments, and fusion polypeptides, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., N.Y., 1987.

In general, a DNA sequence encoding a zins4 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To insure cleavage of the B and A chains, cells transfected with expression vectors containing DNA sequences encoding zins4 are co-transfected with expression vectors encoding a suitable prohormone convertase, for example furin, PC⅓, PC2, PACE, PACE4, or PC4. Such sequences are known in the art and can be inserted into expression vectors and transfected into cells as described herein.

To direct a zins4 polypeptide into the secretory pathway of a host cell, a secretory signal sequence (also known as a leader sequence, prepro sequence or pre sequence) is provided in the expression vector. The secretory signal sequence may be that of zins4, or may be derived from another secreted protein (e.g., t-PA) or synthesized de novo. The secretory signal sequence is operably linked to the zins4 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain secretory signal sequences may be positioned elsewhere in the DNA sequence of interest (see, e.g., Welch et al., U.S. Pat. No. 5,037,743; Holland et al., U.S. Pat. No. 5,143,830).

Alternatively, the secretory signal sequence contained in the polypeptides of the present invention is used to direct other polypeptides into the secretory pathway. The present invention provides for such fusion polypeptides. A signal fusion polypeptide can be made wherein a secretory signal sequence derived from zins4 (amino acid residues 1-25 of SEQ ID NO:2) is operably linked to a DNA sequence encoding another polypeptide using methods known in the art and disclosed herein. The secretory signal sequence contained in the fusion polypeptides of the present invention is preferably fused amino-terminally to an additional peptide to direct the additional peptide into the secretory pathway. Such constructs have numerous applications known in the art. For example, these novel secretory signal sequence fusion constructs can direct the secretion of an active component of a normally non-secreted protein. Such fusions may be used in vivo or in vitro to direct peptides through the secretory pathway.

Cultured mammalian cells are suitable hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-5, 1982), DEAE-dextran mediated transfection (Ausubel et al., ibid.), and liposome-mediated transfection (Hawley-Nelson et al., Focus 15:73, 1993; Ciccarone et al., Focus 15:80, 1993, and viral vectors (Miller and Rosman, BioTechniques 7:980-90, 1989; Wang and Finer, Nature Med. 2:714-6, 1996). The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Manassas, Va. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems can also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used. Alternative markers that introduce an altered phenotype, such as green fluorescent protein, or cell surface proteins such as CD4, CD8, Class I MHC, placental alkaline phosphatase may be used to sort transfected cells from untransfected cells by such means as FACS sorting or magnetic bead separation technology.

Other higher eukaryotic cells can also be used as hosts, including plant cells, insect cells and avian cells. The use of Agrobacterium rhizogenesas a vector for expressing genes in plant cells has been reviewed by Sinkar et al., J. Biosci. (Bangalore) 11:47-58, 1987. Transformation of insect cells and production of foreign polypeptides therein is disclosed by Guarino et al., U.S. Pat. No. 5,162,222 and WIPO publication WO 94/06463. Insect cells can be infected with recombinant baculovirus, commonly derived from Autographa californica nuclear polyhedrosis virus (AcNPV). See, King, and Possee, The Baculovirus Expression System: A Laboratory Guide, London, Chapman & Hall; O'Reilly, et al., Baculovirus Expression Vectors: A Laboratory Manual, New York, Oxford University Press., 1994; and, Richardson, C. D., Ed., Baculovirus Expression Protocols. Methods in Molecular Biology, Totowa, N.J., Humana Press, 1995. The second method of making recombinant baculovirus utilizes a transposon-based system described by Luckow (Luckow, et al., J Virol 67:4566-79, 1993). This system is sold in the Bac-to-Bac™ kit (Life Technologies, Rockville, Md.). This system utilizes a transfer vector, pFastBac1™ (Life Technologies) containing a Tn7 transposon to move the DNA encoding the zins4 polypeptide into a baculovirus genome maintained in E. coli as a large plasmid called a “bacmid.” The pFastBac1™ transfer vector utilizes the AcNPV polyhedrin promoter to drive the expression of the gene of interest, in this case zins4. However, pFastBac1™ can be modified to a considerable degree The polyhedrin promoter can be removed and substituted with the baculovirus basic protein promoter (also known as Pcor, p6.9 or MP promoter) which is expressed earlier in the baculovirus infection, and has been shown to be advantageous for expressing secreted proteins. See, Hill-Perkins and Possee, J. Gen. Virol. 71:971-6, 1990; Bonning, et al., J. Gen. Virol. 75:1551-6, 1994; and, Chazenbalk and Rapoport, J. Biol. Chem. 270:1543-9, 1995. In such transfer vector constructs, a short or long version of the basic protein promoter can be used. Moreover, transfer vectors can be constructed which replace the native zins4 secretory signal sequences with secretory signal sequences derived from insect proteins. For example, a secretory signal sequence from Ecdysteroid Glucosyltransferase (EGT), honey bee Melittin (Invitrogen, Carlsbad, Calif.), or baculovirus gp67 (PharMingen, San Diego, Cailf.) can be used in constructs to replace the native zins4 secretory signal sequence. In addition, transfer vectors can include an in-frame fusion with DNA encoding an epitope tag at the C- or N-terminus of the expressed zins4 polypeptide, for example, a Glu-Glu epitope tag (Grussenmeyer et al., Proc. Natl. Acad. Sci. 82:7952-4, 1985). The tag can then be removed after purification. Using a technique known in the art, a transfer vector containing zins4 is transformed into E. coli, and screened for bacrnids which contain an interrupted lacZ gene indicative of recombinant baculovirus.

The bacmid DNA containing the recombinant baculovirus genome is isolated, using common techniques, and used to transfect Spodoptera frugiperda cells, e.g. Sf9 cells.

Recombinant virus that expresses zins4 is subsequently produced. Recombinant viral stocks are made by methods commonly used the art. The recombinant virus is used to infect host cells, typically a cell line derived from the fall armyworm, Spodoptera frugiperda. See, in general, Glick and Pasternak, Molecular Biotechnoloav:Principles and Applications of Recombinant DNA, ASM Press, Washington, D.C., 1994. Another suitable cell line is the High FiveO™ cell line (Invitrogen) derived from Trichoplusia ni (U.S. Pat. No. 5,300,435). Commercially available serum-free media are used to grow and maintain the cells. Suitable media are Sf900 II™ (Life Technologies) or ESF 921™ (Expression Systems) for the Sf9 cells; and Ex-cellO405™ (JRH Biosciences, Lenexa, KS) or Express FiveO™ (Life Technologies) for the T. ni cells. The cells are grown up from an inoculation density of approximately 2-5×10⁵ cells to a density of 1-2×10⁶ cells at which time a recombinant viral stock is added at a multiplicity of infection (MOI) of 0.1 to 10, more typically near 3. Procedures used are generally described in available laboratory manuals (King and Possee, ibid.; O'Reilly et al., ibid.; Richardson, ibid.).

Subsequent purification of the zins4 polypeptide from the supernatant can be achieved using methods described herein.

Fungal cells, including yeast cells, can also be used within the present invention. Yeast species of particular interest in this regard include Saccharomyces cerevisiae, Pichia pastoris, and Pichia methanolica. Methods for transforming S. cerevisiae cells with exogenous DNA and producing recombinant polypeptides therefrom are disclosed by, for example, Kawasaki, U.S. Pat. No. 4,599,311;

Kawasaki et al., U.S. Pat. No. 4,931,373; Brake, U.S. Pat. No. 4,870,008; Welch et al., U.S. Pat. No. 5,037,743; and Murray et al., U.S. Pat. No. 4,845,075. Transformed cells are selected by phenotype determined by the selectable marker, commonly drug resistance or the ability to grow in the absence of a particular nutrient (e.g., leucine). A preferred vector system for use in Saccharomyces cerevisiae is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092) and alcohol dehydrogenase genes. See also U.S. Pat. Nos. 4,990,446; 5,063,154; 5,139,936 and 4,661,454. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-65, 1986 and Cregg, U.S. Pat. No. 4,882,279. Aspergillus cells may be utilized according to the methods of McKnight et al., U.S. Pat. No. 4,935,349. Methods for transforming Acremonium chrysogenum are disclosed by Sumino et al., U.S. Pat. No. 5,162,228. Methods for transforming Neurospora are disclosed by Lambowitz, U.S. Pat. No. 4,486,533.

The use of Pichia methanolica as host for the production of recombinant proteins is disclosed in WIPO Publications WO 97/17450, WO 97/17451, WO 3 0 98/02536, and WO 98/02565. DNA molecules for use in transforming P. methanolica will commonly be prepared as double-stranded, circular plasmids, which are preferably linearized prior to transformation. For polypeptide production in P. methanolica, it is preferred that the promoter and terminator in the plasmid be that of a P. methanolica gene, such as a P. methanolica alcohol utilization gene (AUG1 or AUG2). Other useful promoters include those of the dihydroxyacetone synthase (DHAS), formate dehydrogenase (FMD), and catalase (CAT) genes. To facilitate integration of the DNA into the host chromosome, it is preferred to have the entire expression segment of the plasmid flanked at both ends by host DNA sequences. A preferred selectable marker for use in Pichia methanolica is a P. methanolica ADE2 gene, which encodes phosphoribosyl-5-aminoimidazole carboxylase (AIRC; EC 4.1.1.21), which allows ade2 host cells to grow in the absence of adenine. For large-scale, industrial processes where it is desirable to minimize the use of methanol, it is preferred to use host cells in which both methanol utilization genes (AUG1 and AUG2) are deleted. For production of secreted proteins, host cells deficient in vacuolar protease genes (PEP4 and PRB1) are preferred. Electroporation is used to facilitate the introduction of a plasmid containing DNA encoding a polypeptide of interest into P. methanolica cells. It is preferred to transform P. methanolica cells by electroporation using an exponentially decaying, pulsed electric field having a field strength of from 2.5 to 4.5 kV/cm, preferably about 3.75 kV/cm, and a time constant (t) of from 1 to 40 milliseconds, most preferably about 20 milliseconds.

Prokaryotic host cells, including strains of the bacteria Escherichia coli, Bacillus and other genera are also useful host cells within the present invention.

Techniques for transforming these hosts and expressing foreign DNA sequences cloned therein are well known in the art (see, e.g., Sambrook et al., ibid.). When expressing a zins4 polypeptide in bacteria such as E. coli, the polypeptide may be retained in the cytoplasm, typically as insoluble granules, or may be directed to the periplasmic space by a bacterial secretion sequence. In the former case, the cells are lysed, and the granules are recovered and denatured using, for example, guanidine isothiocyanate or urea. The denatured polypeptide can then be refolded and dimerized by diluting the denaturant, such as by dialysis against a solution of urea and a combination of reduced and oxidized glutathione, followed by dialysis against a buffered saline solution. In the latter case, the polypeptide can be recovered from the periplasmic space in a soluble and functional form by disrupting the cells (by, for example, sonication or osmotic shock) to release the contents of the periplasmic space and recovering the protein, thereby obviating the need for denaturation and refolding.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell. P. methanolica cells are cultured in a medium comprising adequate sources of carbon, nitrogen and trace nutrients at a temperature of about 25° C. to 35° C. Liquid cultures are provided with sufficient aeration by conventional means, such as shaking of small flasks or sparging of fermentors. A preferred culture medium for P. methanolica is YEPD (2% D-glucose, 2% Bacto™ Peptone (Difco Laboratories, Detroit, Mich.), 1% Bacto™ yeast extract (Difco Laboratories), 0.004% adenine and 0.006% L-leucine).

It is preferred to purify the polypeptides of the present invention to ≧80% purity, more preferably to ≧90% purity, even more preferably ≧95% purity, and particularly preferred is a pharmaceutically pure state, that is greater than 99.9% pure with respect to contaminating macromolecules, particularly other proteins and nucleic acids, and free of infectious and pyrogenic agents. Preferably, a purified polypeptide is substantially free of other polypeptides, particularly other polypeptides of animal origin.

Expressed recombinant zins4 polypeptides (or chimeric zins4 polypeptides) can be purified using fractionation and/or conventional purification methods and media. Ammonium sulfate precipitation and acid or chaotrope extraction may be used for fractionation of samples. Exemplary purification steps can include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Methods for binding receptor polypeptides to support media are well known in the art. Selection of a particular method is a matter of routine design and is determined in part by the properties of the chosen support. See, for example, Affinity Chromatography: Principles & Methods, Pharmacia LKB Biotechnology, Uppsala, Sweden, 1988.

The polypeptides of the present invention can be isolated by exploitation of their structural and biological properties. For example, immobilized metal ion adsorption (IMAC) chromatography can be used to purify histidine-rich proteins, including those comprising polyhistidine tags. Briefly, a gel is first charged with divalent metal ions to form a chelate (Sulkowski, Trends in Biochem. 3:1-7, 1985). Histidine-rich proteins will be adsorbed to this matrix with differing affinities, depending upon the metal ion used, and will be eluted by competitive elution, lowering the pH, or use of strong chelating agents. Other methods of purification include purification of glycosylated proteins by lectin affinity chromatography and ion exchange chromatography (Methods in Enzymol., Vol. 182, “Guide to Protein Purification”, M. Deutscher, (ed.), Acad. Press, San Diego, 1990, pp.529-39).

Within additional embodiments of the invention, a fusion of the polypeptide of interest and an affinity tag (e.g., HIS tag, Glu-Glu, FLAG, maltose-binding protein, an immunoglobulin domain) may be constructed to facilitate purification. Such fusions include polypeptides comprising affinity tags which further comprise a proteolytic cleavage site between the zins4 polypeptide and the affinity tag.

Preferred such sites include thrombin cleavage sites, factor Xa cleavage sites, endoprotease (Arg-Cys or Lys-Cys) sites. To prevent disruption of biological activity, but to facilitate purification, the C peptide can be tagged or replaced.

Additionally, zins4 polypeptides can be purified using prohormone cleavage to isolate the mature protein. The unprocessed polypeptides are first exposed to a prohormone convertase, such as PC ⅓, PC3, PC 4, PACE, PACE4, furin, kex-2, to cleave RR or RXXR sites, followed by cleavage of remaining R residues by a carboxypeptidase.

Moreover, using methods described in the art, polypeptide fusions, or hybrid zins4 proteins, are constructed using regions or domains of zins4 in combination with those of paralogs, orthologs, or heterologous proteins (Sambrook et al., ibid., Altschul et al., ibid., Picard Cur. Opin. Biology 5:511-5, 1994, and references therein). These methods allow the determination of the biological importance of larger domains or regions in a polypeptide of interest. Such hybrids may alter reaction kinetics, binding, constrict or expand the substrate specificity, or alter tissue and cellular localization of a polypeptide, and can be applied to polypeptides of unknown structure.

Fusion polypeptides can be prepared by methods known to those skilled in the art by preparing each component of the fusion protein and chemically conjugating them. Alternatively, a polynucleotide encoding one or more components of the fusion protein in the proper reading frame can be generated using known techniques and expressed by the methods described herein. For example, part or all of a domain(s) conferring a biological function may be swapped between zins4 of the present invention with the functionally equivalent domain(s) from another family member. Such domains include, but are not limited to the secretory signal sequence, the A chain, the B chain and the C peptide, described herein. Such fusion proteins would be expected to have a biological functional profile that is the same or similar to polypeptides of the present invention or other known family proteins or to a heterologous protein, depending on the fusion constructed. Moreover, such fusion proteins may exhibit other properties as disclosed herein.

Standard molecular biological and cloning techniques can be used to swap the equivalent domains between the zins4 polypeptide and those polypeptides to which they are fused. Generally, a DNA segment that encodes a domain of interest, e.g., a zins4 N-terminal polypeptide, the A chain, the B chain, or the C peptide, or motif described herein, is operably linked in frame to at least one other DNA segment encoding an additional polypeptide and inserted into an appropriate expression vector, as described herein. Generally DNA constructs are made such that the several DNA segments that encode the corresponding regions of a polypeptide are operably linked in frame to make a single construct that encodes the entire fusion protein, or a functional portion thereof. For example, a DNA construct would encode from N-terminus to C-terminus a fusion protein comprising a signal polypeptide followed by a mature polypeptide; or a DNA construct would encode from N-terminus to C-terminus a fusion protein comprising a signal polypeptide followed by B chain, C peptide and A chain, or as interchanged with equivalent regions from another protein. Such fusion proteins can be expressed, isolated, and assayed for activity as described herein.

Protein refolding (and optionally reoxidation) procedures may be advantageously used. zins4 polypeptides or fragments thereof may also be prepared through chemical synthesis. zins4 polypeptides may be monomers or multimers;

glycosylated or non-glycosylated; pegylated or non-pegylated; and may or may not include an initial methionine amino acid residue.

Polypeptides of the present invention can also be synthesized by exclusive solid phase synthesis, partial solid phase methods, fragment condensation or classical solution synthesis. Methods for synthesizing polypeptides are well known in the art. See, for example, Merrifield, J. Am. Chem. Soc. 85:2149, 1963; Kaiser et al., Anal. Biochem. 34:595, 1970. After the entire synthesis of the desired peptide on a solid support, the peptide-resin is washed with a reagent which cleaves the polypeptide from the resin and removes most of the side-chain protecting groups. Such methods are well established in the art.

Nucleic acid molecules disclosed herein can be used to detect the expression of a zins4 gene in a biological sample. Such probe molecules include double-stranded nucleic acid molecules comprising the nucleotide sequences of SEQ ID NO:1, or fragments thereof, as well as single-stranded nucleic acid molecules having the complement of the nucleotide sequences of SEQ ID NO:1, or a fragment thereof.

Probe molecules may be DNA, RNA, oligonucleotides, and the like. As an illustration, suitable probes include nucleic acid molecules that bind with a portion of a zins4 A chain, B chain or C peptide, or the sequences of SEQ ID Nos: 3, 4, or 5.

In a basic assay, a single-stranded probe molecule is incubated with RNA, isolated from a biological sample, under conditions of temperature and ionic strength that promote base pairing between the probe and target zins4 RNA species.

After separating unbound probe from hybridized molecules, the amount of hybrids is detected.

Well-established hybridization methods of RNA detection include northern analysis and dot/slot blot hybridization (see, for example, Ausubel ibid. and Wu et al. (eds.), “Analysis of Gene Expression at the RNA Level,” in Methods in Gene Biotechnology, pages 225-239 (CRC Press, Inc. 1997)). Nucleic acid probes can be detectably labeled with radioisotopes such as ³²P or ³⁵S. Alternatively, zins4 RNA can be detected with a nonradioactive hybridization method (see, for example, Isaac (ed.), Protocols for Nucleic Acid Analysis by Nonradioactive Probes, Humana Press, Inc., 1993). Typically, nonradioactive detection is achieved by enzymatic conversion of chromogenic or chemiluminescent substrates. Illustrative nonradioactive moieties include biotin, fluorescein, and digoxigenin.

Zins4 oligonucleotide probes are also useful for in vivo diagnosis. As an illustration, ¹⁸F-labeled oligonucleotides can be administered to a subject and visualized by positron emission tomography (Tavitian et al., Nature Medicine 4:467, 1998).

Numerous diagnostic procedures take advantage of the polymerase chain reaction (PCR) to increase sensitivity of detection methods. Standard techniques for performing PCR are well-known (see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)). PCR primers can be designed to amplify a sequence encoding a particular zins4 domain or motif, such as the A chain, B chain, C peptide, or the sequences of SEQ ID Nos:3, 4, or 5. One variation of PCR for diagnostic assays is reverse transcriptase-PCR (RT-PCR). In the RT-PCR technique, RNA is isolated from a biological sample, reverse transcribed to cDNA, and the cDNA is incubated with zins4 primers (see, for example, Wu et al. (eds.), “Rapid Isolation of Specific cDNAs or Genes by PCR,” in Methods in Gene Biotechnology, CRC Press, Inc., pages 15-28, 1997). PCR is then performed and the products are analyzed using standard techniques.

As an illustration, RNA is isolated from biological sample using, for example, the guanidinium-thiocyanate cell lysis procedure described herein. Alternatively, a solid-phase technique can be used to isolate mRNA from a cell lysate.

A reverse transcription reaction can be primed with the isolated RNA using random oligonucleotides, short homopolymers of dT, or zins4 anti-sense oligomers. Oligo-dT primers offer the advantage that various mRNA nucleotide sequences are amplified that can provide control target sequences. Zins4 sequences are amplified by the polymerase chain reaction using two flanking oligonucleotide primers that are typically at least 5 bases in length.

PCR amplification products can be detected using a variety of approaches. For example, PCR products can be fractionated by gel electrophoresis, and visualized by ethidium bromide staining. Alternatively, fractionated PCR products can be transferred to a membrane, hybridized with a detectably-labeled zins4 probe, and examined by autoradiography. Additional alternative approaches include the use of digoxigenin-labeled deoxyribonucleic acid triphosphates to provide chemiluminescence detection, and the C-TRAK colorimetric assay.

Another approach is real time quantitative PCR (Perkin-Elmer Cetus, Norwalk, Ct.). A fluorogenic probe, consisting of an oligonucleotide with both a reporter and a quencher dye attached, anneals specifically between the forward and reverse primers. Using the 5′ endonuclease activity of Taq DNA polymerase, the reporter dye is separated from the quencher dye and a sequence-specific signal is generated and increases as amplification increases. The fluorescence intensity can be continuously monitored and quantified during the PCR reaction.

Another approach for detection of zins4 expression is cycling probe technology (CPT), in which a single-stranded DNA target binds with an excess of DNA-RNA-DNA chimeric probe to form a complex, the RNA portion is cleaved with RNase H, and the presence of cleaved chimeric probe is detected (see, for example, Beggs et al., J. Clin. Microbiol. 34:2985, 1996 and Bekkaoui et al., Biotechniques 20:240, 1996). Alternative methods for detection of zins4 sequences can utilize approaches such as nucleic acid sequence-based amplification (NASBA), cooperative amplification of templates by cross-hybridization (CATCH), and the ligase chain reaction (LCR) (see, for example, Marshall et al., U.S. Pat. No. 5,686,272 (1997), Dyer et al., J. Virol. Methods 60:161, 1996; Ehricht et al., Eur. J. Biochem. 243:358, 1997 and Chadwick et al., J. Virol. Methods 70:59, 1998). Other standard methods are known to those of skill in the art.

Zins4 probes and primers can also be used to detect and to localize zins4 gene expression in tissue samples. Methods for such in situ hybridization are well-known to those of skill in the art (see, for example, Choo (ed.), In Situ Hybridization Protocols, Humana Press, Inc., 1994; Wu et al. (eds.), “Analysis of Cellular DNA or Abundance of mRNA by Radioactive In Situ Hybridization (RISH),” in Methods in Gene Biotechnology, CRC Press, Inc., pages 259-278, 1997 and Wu et al. (eds.), “Localization of DNA or Abundance of MRNA by Fluorescence In Situ Hybridization (RISH),” in Methods in Gene Biotechnology, CRC Press, Inc., pages 279-289, 1997).

Various additional diagnostic approaches are well-known to those of skill in the art (see, for example, Mathew (ed.), Protocols in Human Molecular Genetics Humana Press, Inc., 1991; Coleman and Tsongalis, Molecular Diagnostics, Humana Press, Inc., 1996 and Elles, Molecular Diagnosis of Genetic Diseases, Humana Press, Inc., 1996).

Polypeptides associated with the ovaries are useful for modulating steroidogenesis, both in vivo and in vitro, and modulating aspects of the ovarian cycle such as oocyte maturation, ovarian cell-cell interactions, follicular development and rupture, luteal function, promoting uterine implantation of fertilized oocytes as well as modulating activities associated the pregnancy, such as gestation and labor, including the force and rate of contractions. Imbalances in reproductive polypeptides can be used to assess the existence of diseases, as well as determine whether normal hormonal balance has been restored after administration of a therapeutic agent. Determination of estradiol, progesterone, LH, and FSH, for example, from serum is known by one of skill in the art. Such assays can be used to monitor hormone levels after administration of zins4 in vivo, or in a transgenic mouse model where the zins4 gene is expressed or the murine ortholog is deleted. Zins4 polypeptides can be used alone or in combination with other reproductive proteins such as relaxin, as a therapeutic application for pregnancy support.

Proteins of the present invention may also be used in applications for enhancing fertilization during assisted reproduction in humans and in animals. Such assisted reproduction methods are known in the art and include artificial insemination, in vitro fertilization, embryo transfer, and gamete intrafallopian transfer. Such methods are useful for assisting those who may have physiological or metabolic disorders that prevent or impede natural conception. Such methods are also used in animal breeding programs, e.g., for livestock, racehorses, domestic and wild animals, and could be used as methods for the creation of transgenic animals.

Proteins of the present invention may also be useful in therapies for treating reproductive disorders. Disorders such as luteal phase deficiency would benefit from such therapy (Soules, “Luteal phase deficiency: A subtle abnormality of ovulation” in, Infertility: Evaluation and Treatment, Keye et al., eds., Philadelphia, WB Saunders, 1995). Administration of reproductive hormones, such as gonadotropin-releasing hormone, is shown to stimulate reproductive behavior (Riskin and Moss, Res. Bull. 11:481-5, 1983; Kadar et al., Physiol. Behav. 51:601-5, 1992 and Silver et al., J. Neruoendoctin. 4:207-10, 199; King and Millar, Cell. Mol. Neurobiol. 15:5-23, 1995). Given the high prevalence of sexual dysfunction and impotence in humans, molecules, polypeptides which may modulate or enhance reproductive activity can find application in developing treatments for these conditions.

Proteins associated with the ovaries may modulate hormones, hormone receptors, growth factors, or cell-cell interactions, of the reproductive cascade or have an involvement in oocyte or ovarian development. The polypeptides, nucleic acid and/or antibodies of the present invention may be used in treatment of disorders associated with gonadal development, pregnancy, pubertal changes, menopause, ovarian cancer, fertility, ovarian function, polycystic ovarian syndrome and other reproductive functions. The molecules of the present invention may used to modulate or to treat or prevent development of pathological conditions in ovary. In particular, certain syndromes or diseases may be amenable to such diagnosis, treatment or prevention. Moreover, natural functions, such as ovulation, may be suppressed or controlled for use in birth control by molecules of the present invention.

Diagnostic methods of the present invention involve the detection of zins4 polypeptides in the serum or tissue biopsy of a patient undergoing analysis of reproductive function or evaluation for possible ovarian cancer. Such polypeptides can be detected using immunoassay techniques and antibodies, described herein, that are capable of recognizing polypeptide epitopes. Such methods include using probes or primers derived, for example, from the nucleotide sequences disclosed herein to detect zins4 expression in a patient sample, such as a blood, saliva, sweat, tissue sample, or the like. For example, probes can be hybridized to tumor tissues and the hybridized complex detected by in situ hybridization. Zins4 sequences can also be detected by PCR amplification using cDNA generated by reverse translation of sample MRNA as a template (PCR Primer A Laboratory Manual, Dieffenbach and Dveksler, eds., Cold Spring Harbor Press, 1995). When compared with a normal control, both increases or decreases of zins4 expression in a patient sample, relative to that of a control, can be monitored and used as an indicator or diagnostic for disease.

The polypeptides of the present invention may modulate contractility in certain tissues. To identify such activity in zins4, assays known in the art can be applied to tissue samples, such as aortic rings, vas deferens, ileum, uterine and other contractile tissue samples, as well as to organ systems, such as atria, and can be used to determine whether zins4 polypeptide, its agonists or antagonists, enhance or depress contractility. Molecules of the present invention are hence useful for treating dysfunction associated with contractile tissues or can be used to suppress or enhance contractility in vivo. As such, molecules of the present invention have utility in treating cardiovascular disease, infertility, in vitro fertilization, birth control, treating impotence or other male reproductive dysfunction, as well as inducing birth.

The effect of the zins4 polypeptides, antagonists and agonists of the present invention on contractility of tissues can be measured in a tensiometer that measures contractility and relaxation in tissues. See, Dainty et al., J. Pharmacol. 100:767, 1990; Rhee et al., Neurotox. 16: 179, 1995; Anderson, Endocrinol. 114:364-8, 1984; and Downing and Sherwood, Endocrinol. 116:1206-14, 1985. For example, measuring vasodilatation of aortic rings is well known in the art. Briefly, aortic rings are taken from 4 month old Sprague Dawley rats and placed in a buffer solution, such as modified Krebs solution (118.5 mnM NaCl, 4.6 mM KCl, 1.2 mM MgSO₄.7H₂O, 1.2 mM KH₂PO₄, 2.5 mM CaCl₂.2H₂O, 24.8 mM NaHCO₃ and 10 mM glucose). One of skill in the art would recognize that this method can be used with other animals, such as rabbits, other rat strains, Guinea pigs, and the like. The rings are then attached to an isometric force transducer (Radnoti Inc., Monrovia, Calif.) and the data recorded with a Ponemah physiology platform (Gould Instrument systems, Inc., Valley View, Ohio) and placed in an oxygenated (95% O₂, 5% CO₂) tissue bath containing the buffer solution. The tissues are adjusted to 1 gram resting tension and allowed to stabilize for about one hour before testing. The integrity of the rings can be tested with norepinepherin (Sigma Co., St. Louis, Mo.) and Carbachol, a muscarinic acetylcholine agonist (Sigma Co.). After integrity is checked, the rings are washed three times with fresh buffer and allowed to rest for about one hour. To test a sample for vasodilatation, or relaxation of the aortic ring tissue, the rings are contracted to two grams tension and allowed to stabilize for fifteen minutes. A zins4 polypeptide sample is then added to 1, 2 or 3 of the 4 baths, without flushing, and tension on the rings recorded and compared to the control rings containing buffer only. Enhancement or relaxation of contractility by zins4 polypeptides, their agonists and antagonists is directly measured by this method, and it can be applied to other contractile tissues such as uterus, prostate, and testis.

Zins4 could be useful as modulator of blood pressure, muscle tension or and osmotic balance. For example, blood pressure modification is important in situations such as heart attack, stroke, traumatic shock, surgery, and any number of bleeding complications. As a modulator of blood pressure, muscle tension or and osmotic balance, zins4 may modulate contractility in the organ systems and tissues that it effects. Thus, the activity of molecules of the present invention can be measured using a variety of assays that measure cell contractility and discussed below. Such assays are well known in the art, and described herein.

Zins4 may also have CNS functions as well as cardiovascular functions. Assays and models to test for such zins4 activity are well known in the art and described herein. For example, see Feng et al., Acta. Physiol. Scand. 166:285-91, 1999; (pithed rat heart failure model to assess vascular sympathetic nerve activity); Horackova, et al., Cell Tissue Res. 297:409-21, 1999 (guinea pig atria model); McLean et al., Neuroscience 92:1377-87, 1999 (CNS response to hypotensive challenge to assess neuron response or activation within cardiovascular control); Potter, et al; Regul. Pept. 25:167-77, 1989 (Testing effects of polypeptides and peptide fragments on blood pressure and vagal action at the heart); Maturi, et al., J. Clin. Invest 83:1217-24 (myocardial ischemia and coronary constriction model in dogs); Haass et al., Naunyn Schmiedebergs Arch. PharmacoL 339:71-8, 1989 (pre-synaptic modulation in in situ perfused guinea pig heart); Hassall, and Bumstock, Neurosci. Lett. 52:111-5, 1984 (cultured guinea pig atria to study intrinsic innervation); Lundberg, et al., Acta. Physiol. Scand. 121:325-32, 1984 (effect of peptide on muscle tone, and autonomic transmission in Guinea pig atrium, vas deferens, urinary bladder, portal vein, and trachea); Miyata et al., Ann. N.Y. Acad. Sci. 865:73-81, 1998 (effect of peptides on rat aortic smooth muscle cell proliferation); Saita et al., Am. J. Physiol. 274:R979-84, 1998 (Effects of centrally administered peptide on blood pressure, heart rate, renal sympathetic nerve activity in rats); Hall et al., Brain Res. 497:280-90, 1989 (microinjection of peptides into the nucleus of the solitary tract (NTS) and effects on cardiovascular function).

The activity of molecules of the present invention can be measured using a variety of assays that measure cell differentiation and proliferation as well as assays that measure cell contractility and cardiovascular function. Such assays are well known in the art.

The molecules of the present invention are useful as components of defined cell culture media, as described herein, and may be used alone or in combination with other cytokines and hormones to replace serum that is commonly used in cell culture. Molecules of the present invention are particularly useful in specifically promoting the growth, development, differentiation, and/or maturation of ovarian cells in culture, and may also prove useful in the study of the ovarian cycle, reproductive function, ovarian cell-cell interactions, and fertilization. Additionally the molecules of the present invention may be useful for promoting the growth and/or development of myocytes in culture, such as cardiac myocytes or myoblasts; skeletal myocytes or myoblasts and smooth muscle cells; chrondrocytes; endothelial cells; adipocytes and osteoblasts in vitro. For example, molecules of the present invention are useful as components of defined cell culture media, and may be used alone or in combination with other cytokines and hormones to replace serum that is commonly used in cell culture, and may also prove useful in the study of cardiac myocyte hyperplasia and regeneration.

Proteins of the present invention are useful for example, in treating reproductive, prostate, heart, kidney and other disorders, and can be measured in vitro using cultured cells or in vivo by administering molecules of the present invention to the appropriate animal model. An in vivo approach for assaying proteins of the present invention involves viral delivery systems. Exemplary viruses for this purpose include adenovirus, herpesvirus, retroviruses, vaccinia virus, and adeno-associated virus (AAV). Adenovirus, a double-stranded DNA virus, is currently the best studied gene transfer vector for delivery of heterologous nucleic acid (for review, see Becker et al., Meth. Cell Biol. 43:161-89, 1994; and Douglas and Curiel, Science & Medicine 4:44-53, 1997). The adenovirus system offers several advantages: (i) adenovirus can accommodate relatively large DNA inserts; (ii) can be grown to high-titer; (iii) infect a broad range of mammalian cell types; and (iv) can be used with many different promoters including ubiquitous, tissue specific, and regulatable promoters. Also, because adenoviruses are stable in the bloodstream, they can be administered by intravenous injection.

Using adenovirus vectors where portions of the adenovirus genome are deleted, inserts are incorporated into the viral DNA by direct ligation or by homologous recombination with a co-transfected plasmid. In an exemplary system, the essential El gene has been deleted from the viral vector, and the virus will not replicate unless the El gene is provided by the host cell (the human 293 cell line is exemplary). When intravenously administered to intact animals, adenovirus primarily targets the liver. If the adenoviral delivery system has an E1 gene deletion, the virus cannot replicate in the host cells. However, the host's tissue (e.g., liver) will express and process (and, if a secretory signal sequence is present, secrete) the heterologous protein. Secreted proteins will enter the circulation in the highly vascularized liver, and effects on the infected animal can be determined.

Moreover, adenoviral vectors containing various deletions of viral genes can be used in an attempt to reduce or eliminate immune responses to the vector. Such adenoviruses are E1 deleted, and in addition contain deletions of E2A or E4 (Lusky, et al., J. Virol. 72:2022-32, 1998; Raper, et al., Human Gene Therapy 9:671-9, 1998). In addition, deletion of E2b is reported to reduce immune responses (Amalfitano, et al., J. Virol. 72:926-33, 1998). Moreover, by deleting the entire adenovirus genome, very large inserts of heterologous DNA can be accommodated. Generation of so called “gutless” adenoviruses where all viral genes are deleted are particularly advantageous for insertion of large inserts of heterologous DNA. For review, see Yeh and Perricaudet., FASEB J. 11:615-23, 1997.

The adenovirus system can also be used for protein production in vitro.

By culturing adenovirus-infected non-293 cells under conditions where the cells are not rapidly dividing, the cells can produce proteins for extended periods of time. For instance, BHK cells are grown to confluence in cell factories, then exposed to the adenoviral vector encoding the secreted protein of interest. The cells are then grown under serum-free conditions, which allows infected cells to survive for several weeks without significant cell division. Alternatively, adenovirus vector infected 293 cells can be grown as adherent cells or in suspension culture at relatively high cell density to produce significant amounts of protein (See Gamier et al., Cytotechnol. 15:145-55, 1994). With either protocol, an expressed, secreted heterologous protein can be repeatedly isolated from the cell culture supernatant, lysate, or membrane fractions depending on the disposition of the expressed protein in the cell. Within the infected 293 cell production protocol, non-secreted proteins may also be effectively obtained.

As a ligand, the activity of zins4 polypeptide can be measured by a silicon-based biosensor microphysiometer which measures the extracellular acidification rate or proton excretion associated with receptor binding and subsequent physiologic cellular responses. An exemplary device is the Cytosensor™ Microphysiometer manufactured by Molecular Devices, Sunnyvale, Calif. A variety of cellular responses, such as cell proliferation, ion transport, energy production, inflammatory response, regulatory and receptor activation, and the like, can be measured by this method. See, for example, McConnell et al., Science 257:1906-12, 1992; Pitchford et al., Meth. Enzymol. 228:84-108, 1997; Arimilli et al., J. Immunol. Meth. 212:49-59, 1998; Van Liefde et al., Eur. J. Phannacol. 346:87-95, 1998. The microphysiometer can be used for assaying adherent or non-adherent eukaryotic or prokaryotic cells. By measuring extracellular acidification changes in cell media over time, the microphysiometer directly measures cellular responses to various stimuli, including zins4 polypeptide, its agonists, or antagonists. Preferably, the microphysiometer is used to measure responses of a zins4-responsive eukaryotic cell, compared to a control eukaryotic cell that does not respond to zins4 polypeptide. Zins4-responsive eukaryotic cells comprise cells into which a receptor for zins4 has been transfected creating a cell that is responsive to zins4; or cells naturally responsive to zins4 such as cells derived from prostate, testis, uterine tissue, or the like. Differences, measured by a change, for example, an increase or diminution in extracellular acidification, in the response of cells exposed to zins4 polypeptide, relative to a control not exposed to zins4, are a direct measurement of zins4-modulated cellular responses. Moreover, such zins4-modulated responses can be assayed under a variety of stimuli. Using the microphysiometer, there is provided a method of identifying agonists of zins4 polypeptide, comprising providing cells responsive to a zins4 polypeptide, culturing a first portion of the cells in the absence of a test compound, culturing a second portion of the cells in the presence of a test compound, and detecting a change, for example, an increase or diminution, in a cellular response of the second portion of the cells as compared to the first portion of the cells. The change in cellular response is shown as a measurable change extracellular acidification rate. Moreover, culturing a third portion of the cells in the presence of zins4 polypeptide and the absence of a test compound can be used as a positive control for the zins4-responsive cells, and as a control to compare the agonist activity of a test compound with that of the zins4 polypeptide. Moreover, using the microphysiometer, there is provided a method of identifying antagonists of zins4 polypeptide, comprising providing cells responsive to a zins4 polypeptide, culturing a first portion of the cells in the presence of zins4 and the absence of a test compound, culturing a second portion of the cells in the presence of zins4 and the presence of a test compound, and detecting a change, for example, an increase or a diminution in a cellular response of the second portion of the cells as compared to the first portion of the cells. The change in cellular response is shown as a measurable change extracellular acidification rate. Antagonists and agonists, for zins4 polypeptide, can be rapidly identified using this method.

Moreover, zins4 can be used to identify cells, tissues, or cell lines which respond to a zins4-stimulated pathway. The microphysiometer, described above, can be used to rapidly identify ligand-responsive cells, such as cells responsive to zins4 of the present invention. Cells can be cultured in the presence or absence of zins4 polypeptide. Those cells which elicit a measurable change in extracellular acidification in the presence of zins4 are responsive to zins4. Such cell lines, can be used to identify antagonists and agonists of zins4 polypeptide as described above.

In view of the tissue distribution observed for zins4 polypeptides, agonists (including the natural ligand/substrate/cofactor/etc.) and antagonists have enormous potential in both in vitro and in vivo applications. For example, zins4 polypeptide and agonist compounds are useful as components of defined cell culture media, and may be used alone or in combination with cytokines and hormones to replace serum that is commonly used in cell culture. Agonists are thus useful in specifically promoting the growth and/or development of mammalian cells in vitro, particularly of those derived from reproductive tissues. As such, zins4 polypeptides or agonists are added to tissue culture media for these cell types.

Zins4 can also be used to identify inhibitors (antagonists) of its activity. Test compounds are added to assays disclosed herein to identify compounds that inhibit the activity of zins4. In addition to those assays disclosed herein, samples can be tested for inhibition of zins4 activity within a variety of assays designed to measure receptor binding or the stimulation/inhibition of zins4-dependent cellular responses. For example, zins4-responsive cell lines can be transfected with a reporter gene construct that is responsive to a zins4-stimulated cellular pathway. Reporter gene constructs of this type are known in the art, and will generally comprise a zins4-DNA response element operably linked to a gene encoding an assayable protein, such as luciferase. DNA response elements can include, but are not limited to, cyclic AMP response elements (CRE), hormone response elements (HRE) insulin response element (IRE) (Nasrin et al., Proc. Natl. Acad. Sci. USA 87:5273-7, 1990) and serum response elements (SRE) (Shaw et al. Cell 56: 563-72, 1989). Cyclic AMP response elements are reviewed in Roestler et al., J. Biol. Chem. 263 (19):9063-6; 1988 and Habener, Molec. Endocrinol. 4 (8):1087-94; 1990. Hormone response elements are reviewed in Beato, Cell 56:33544; 1989. Candidate compounds, solutions, mixtures or extracts are tested for the ability to inhibit the activity of zins4 on the target cells as evidenced by a decrease in zins4 stimulation of reporter gene expression. Assays of this type will detect compounds that directly block zins4 binding to cell-surface receptors, as well as compounds that block processes in the cellular pathway subsequent to receptor-ligand binding. In the alternative, compounds or other samples can be tested for direct blocking of zins4 binding to receptor using zins4 tagged with a detectable label (e.g., ¹²⁵I, biotin, horseradish peroxidase, HFRC, and the like). Within assays of this type, the ability of a test sample to inhibit the binding of labeled zins4 to the receptor is indicative of inhibitory activity, which can be confirmed through secondary assays. Receptors used within binding assays may be cellular receptors or isolated, immobilized receptors.

A zins4 ligand-binding polypeptide can also be used for purification of ligand. The polypeptide is immobilized on a solid support, such as agarose beads, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting medium will generally be configured in the form of a column, and fluids containing ligand are passed through the column one or more times to allow ligand to bind to the receptor polypeptide. The ligand is then eluted using changes in salt concentration, chaotropic agents (guanidine HCl), or pH to disrupt ligand-receptor binding.

An assay system that uses a ligand-binding receptor (or an antibody, one member of a complement/ anti-complement pair) or a binding fragment thereof, and a commercially available biosensor instrument (BIAcore, Pharmacia Biosensor, Piscataway, N.J.) may be advantageously employed. Such receptor, antibody, member of a complement/anti-complement pair or fragment is immobilized onto the surface of a receptor chip. Use of this instrument is disclosed by Karlsson, J. Immunol. Methods 145:229-40, 1991 and Cunningham and Wells, J. Mol. Biol. 234:554-63, 1993. A receptor, antibody, member or fragment is covalently attached, using amine or sulfhydryl chemistry, to dextran fibers that are attached to gold film within the flow cell. A test sample is passed through the cell. If a ligand, epitope, or opposite member of the complement/anti-complement pair is present in the sample, it will bind to the immobilized receptor, antibody or member, respectively, causing a change in the refractive index of the medium, which is detected as a change in surface plasmon resonance of the gold film. This system allows the determination of on- and off-rates, from which binding affinity can be calculated, and assessment of stoichiometry of binding.

Ligand-binding receptor polypeptides can also be used within other assay systems known in the art. Such systems include Scatchard analysis for determination of binding affinity (see Scatchard, Ann. NY Acad. Sci. 51: 660-72, 1949) and calorimetric assays (Cunningham et al., Science 253:54548, 1991; Cunningham et al., Science 245:821-25, 1991).

Zins4 polypeptides can also be used to prepare antibodies that bind to zins4 epitopes, peptides or polypeptides. The zins4 polypeptide or a fragment thereof serves as an antigen (immunogen) to inoculate an animal and elicit an immune response. One of skill in the art would recognize that antigenic, epitope-bearing polypeptides contain a sequence of at least 6, preferably at least 9, and more preferably at least 15 to about 30 contiguous amino acid residues of a zins4 polypeptide (e.g., SEQ ID NO:2). Polypeptides comprising a larger portion of a zins4 polypeptide, i.e., from to 30 residues up to the entire length of the amino acid sequence are included.

Antigens or immunogenic epitopes can also include attached tags, adjuvants and carriers, as described herein. Suitable antigens include the zins4 polypeptide encoded by SEQ ID NO:2 from amino acid number 1 (Met) to amino acid number 142 (Cys), or a contiguous 9 to 142 amino acid fragment thereof. Other suitable antigens include the signal sequence, the A chain, the B chain and the C peptide, as disclosed herein. Still other suitable antigens include, but are not limited to, polypeptides from amino acid residue 25-52, 26-52, 26-53, 26-54, 55-114, 55-115, 55-116, 55-117, 55-118, 26-114, 55-142, 1-25, 1-52, 26-54, 1-118, 119-142, and 26-142 of SEQ ID NO:2. Other peptides to use as antigens are hydrophilic peptides such as those predicted by one of skill in the art from a hydrophobicity plot. Antibodies from an immune response generated by inoculation of an animal with these antigens can be isolated and purified as described herein. Methods for preparing and isolating polyclonal and monoclonal antibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratorv Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982.

As would be evident to one of ordinary skill in the art, polyclonal antibodies can be generated from inoculating a variety of warm-blooded animals such as horses, cows, goats, sheep, dogs, chickens, rabbits, mice, and rats with a zins4 polypeptide or a fragment thereof. The immunogenicity of a zins4 polypeptide may be increased through the use of an adjuvant, such as alum (aluminum hydroxide) or Freund's complete or incomplete adjuvant. Polypeptides useful for immunization also include fusion polypeptides, such as fusions of zins4 or a portion thereof with an immunoglobulin polypeptide or with maltose binding protein. The polypeptide immunogen may be a full-length molecule or a portion thereof. If the polypeptide portion is “hapten-like”, such portion may be advantageously joined or linked to a macromolecular carrier (such as keyhole limpet hemocyanin (KLH), bovine serum albumin (BSA) or tetanus toxoid) for immunization.

As used herein, the term “antibodies” includes polyclonal antibodies, affinity-purified polyclonal antibodies, monoclonal antibodies, and antigen-binding fragments, such as F(ab′)₂ and Fab proteolytic fragments. Genetically engineered intact antibodies or fragments, such as chimeric antibodies, Fv fragments, single chain antibodies and the like, as well as synthetic antigen-binding peptides and polypeptides, are also included. Non-human antibodies may be humanized by grafting non-human CDRs onto human framework and constant regions, or by incorporating the entire non-human variable domains (optionally “cloaking” them with a human-like surface by replacement of exposed residues, wherein the result is a “veneered” antibody). In some instances, humanized antibodies may retain non-human residues within the human variable region framework domains to enhance proper binding characteristics. Through humanizing antibodies, biological half-life may be increased, and the potential for adverse immune reactions upon administration to humans is reduced.

For particular uses, it may be desirable to prepare fragments of anti-zins4 antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 89:230, 1960, Porter, Biochem. J. 73:119, 1959, Edelman et al., in Methods in Enzvmologv Vol. 1, page 422 (Academic Press 1967), and by Coligan, ibid.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Natl. Acad. Sci. USA 69:2659, 1972. Alternatively, the variable chains can be linked by an intermolecular disulfide bond or cross-linked by chemicals such as gluteraldehyde (see, for example, Sandhu, Crit. Rev. Biotech. 12:437, 1992).

The Fv fragments may comprise V_{H} and V_{L} chains which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97, 1991, also see, Bird et al., Science 242:423, 1988, Ladner et al., U.S. Pat. No. 4,946,778, Pack et al., Bio/Technology 11:1271, 1993, and Sandhu, ibid.

As an illustration, a scFV can be obtained by exposing lymphocytes to zins4 polypeptide in vitro, and selecting antibody display libraries in phage or similar vectors (for instance, through use of immobilized or labeled zins4 protein or peptide).

Genes encoding polypeptides having potential zins4 polypeptide binding domains can be obtained by screening random peptide libraries displayed on phage (phage display) or on bacteria, such as E. coli. Nucdeotide sequences encoding the polypeptides can be obtained in a number of ways, such as through random mutagenesis and random polynucleotide synthesis. These random peptide display libraries can be used to screen for peptides which interact with a known target which can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Disiplay of Peptides and Proteins (Academic Press, Inc. 1996)) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the zins4 sequences disclosed herein to identify proteins which bind to zins4.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106, 1991), Courtenay-Luck, “Genetic Manipulation of Monoclonal Antibodies,” in Monoclonal Antibodies: Production, Engineering and Clinical Application, Ritter et al. (eds.), page 166 (Cambridge University Press, 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternative techniques for generating or selecting antibodies useful herein include in vitro exposure of lymphocytes to zins4 protein or peptide, and selection of antibody display libraries in phage or similar vectors (for instance, through use of immobilized or labeled zins4 protein or peptide). Genes encoding polypeptides having potential zins4 polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides which-interact with a known target which can be a protein or polypeptide, such as a ligand or receptor, a biological or synthetic macromolecule, or organic or inorganic substances.

Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially, for instance from Clontech (Palo Alto, Calif.), Invitrogen Inc. (San Diego, Calif.), New England Biolabs, Inc. (Beverly, Mass.) and Pharmacia LKB Biotechnology Inc. (Piscataway, N.J.). Random peptide display libraries can be screened using the zins4 sequences disclosed herein to identify proteins which bind to zins4.

These “binding polypeptides” which interact with zins4 polypeptides can be used for tagging cells; for isolating homolog polypeptides by affinity purification; they can be directly or indirectly conjugated to drugs, toxins, radionuclides and the like. These binding polypeptides can also be used in analytical methods such as for screening expression libraries and neutralizing activity, e.g., for blocking interaction between ligand and receptor, or viral binding to a receptor. The binding polypeptides can also be used for diagnostic assays for determining circulating levels of zins4 polypeptides; for detecting or quantitating soluble zins4 polypeptides as marker of underlying pathology or disease. These binding polypeptides can also act as zins4 “antagonists” to block zins4 binding and signal transduction in vitro and in vivo. These anti-zins4 binding polypeptides would be useful for inhibiting zins4 activity or protein-binding.

Antibodies are considered to be specifically binding if: 1) they exhibit a threshold level of binding activity, and 2) they do not significantly cross-react with related polypeptide molecules. A threshold level of binding is determined if anti-zins4 antibodies herein bind to a zins4 polypeptide, peptide or epitope with an affinity at least 10-fold greater than the binding affinity to control (non-zins4) polypeptide. It is preferred that the antibodies exhibit a binding affinity (Kₐ) of 10⁶ M⁻¹ or greater, preferably 10⁷ M⁻¹ or greater, more preferably 10⁸ M⁻¹ or greater, and most preferably 10 M⁻¹ or greater. The binding affinity of an antibody can be readily determined by one of ordinary skill in the art, for example, by Scatchard analysis (Scatchard, G., Ann. NY Acad. Sci. 51: 660-672, 1949).

Whether anti-zins4 antibodies do not significantly cross-react with related polypeptide molecules is shown, for example, by the antibody detecting zins4 polypeptide but not known related polypeptides using a standard Western blot analysis (Ausubel et al., ibid.). Examples of known related polypeptides are those disclosed in the prior art, such as known orthologs, and paralogs, and similar known members of a protein family, Screening can also be done using non-human zins4, and zins4 mutant polypeptides. Moreover, antibodies can be “screened against” known related polypeptides, to isolate a population that specifically binds to the zins4 polypeptides. For example, antibodies raised to zins4 are adsorbed to related polypeptides adhered to insoluble matrix; antibodies specific to zins4 will flow through the matrix under the proper buffer conditions. Screening allows isolation of polyclonal and monoclonal antibodies non-crossreactive to known closely related polypeptides (Antibodies: A Laboratory Manual, Harlow and Lane (eds.), Cold Spring Harbor Laboratory Press, 1988; Current Protocols in Immunology, Cooligan, et al. (eds.), National Institutes of Health, John Wiley and Sons, Inc., 1995). Screening and isolation of specific antibodies is well known in the art. See, Fundamental lmmunology, Paul (eds.), Raven Press, 1993; Getzoff et al., Adv. in ImmunoL 43: 1-98, 1988; Monoclonal Antibodies: Principles and Practice, Goding, J. W. (eds.), Academic Press Ltd., 1996; Benjamin et al., Ann. Rev. Immunol. 2: 67-101, 1984. Specifically binding anti-zins4 antibodies can be detected by a number of methods in the art, and disclosed below.

A variety of assays known to those skilled in the art can be utilized to detect antibodies which bind to zins4 proteins or polypeptides. Exemplary assays are described in detail in Antibodies: A Laboratorv Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assay, inhibition or competition assay, and sandwich assay. In addition, antibodies can be screened for binding to wild-type versus mutant zins4 protein or polypeptide.

Antibodies to zins4 may be used for tagging cells that express zins4; for isolating zins4 by affinity purification; for diagnostic assays for determining circulating levels of zins4 polypeptides; for detecting or quantitating soluble zins4 as marker of underlying pathology or disease; in analytical methods employing FACS; for screening expression libraries; for generating anti-idiotypic antibodies; and as neutralizing antibodies or as antagonists to block zins4 activity in vitro and in vivo. Suitable direct tags or labels include radionuclides, enzymes, substrates, cofactors, inhibitors, fluorescent markers, chemiluminescent markers, magnetic particles and the like;

indirect tags or labels may feature use of biotin-avidin or other complement/anti-complement pairs as intermediates. Antibodies herein may also be directly or indirectly conjugated to drugs, toxins, radionuclides and the like, and these conjugates used for in vivo diagnostic or therapeutic applications. Moreover, antibodies to zins4 or fragments thereof may be used in vitro to detect denatured zins4 or fragments thereof in assays, for example, Western Blots or other assays known in the art.

Antibodies or polypeptides herein can also be directly or indirectly conjugated to drugs, toxins, radionuclides and the like, and these conjugates used for in vivo diagnostic or therapeutic applications. For instance, polypeptides or antibodies of the present invention can be used to identify or treat tissues or organs that express a corresponding anti-complementary molecule (receptor or antigen, respectively, for instance). More specifically, zins4 polypeptides or anti-zins4 antibodies, or bioactive fragments or portions thereof, can be coupled to detectable or cytotoxic molecules and delivered to a mammal having cells, tissues or organs that express the anti-complementary molecule.

Suitable detectable molecules may be directly or indirectly attached to the polypeptide or antibody, and include radionuclides, enzymes, substrates, cofactors, inhibitors, fluorescent markers, chemiluminescent markers, magnetic particles and the like. Suitable cytotoxic molecules may be directly or indirectly attached to the polypeptide or antibody, and include bacterial or plant toxins (for instance, diphtheria toxin, Pseudomonas exotoxin, ricin, abrin and the like), as well as therapeutic radionuclides, such as iodine-131, rhenium-188 or yttrium-90 (either directly attached to the polypeptide or antibody, or indirectly attached through means of a chelating moiety, for instance). Polypeptides or antibodies may also be conjugated to cytotoxic drugs, such as adriamycin. For indirect attachment of a detectable or cytotoxic molecule, the detectable or cytotoxic molecule can be conjugated with a member of a complementary/ anticomplementary pair, where the other member is bound to the polypeptide or antibody portion. For these purposes, biotin/streptavidin is an exemplary complementary/ anticomplementary pair.

In another embodiment, polypeptide-toxin fusion proteins or antibody-toxin fusion proteins can be used for targeted cell or tissue inhibition or ablation (for instance, to treat cancer cells or tissues). Alternatively, if the polypeptide has multiple functional domains (i.e., an activation domain or a receptor binding domain, plus a targeting domain), a fusion protein including only the targeting domain may be suitable for directing a detectable molecule, a cytotoxic molecule or a complementary molecule to a cell or tissue type of interest. In instances where the domain only fusion protein includes a complementary molecule, the anti-complementary molecule can be conjugated to a detectable or cytotoxic molecule. Such domain-complementary molecule fusion proteins thus represent a generic targeting vehicle for cell/tissue-specific delivery of generic anti-complementary-detectable/ cytotoxic molecule conjugates.

The bioactive polypeptide or antibody conjugates described herein can be delivered intravenously, intraarterially or intraductally, or may be introduced locally at the intended site of action.

Molecules of the present invention can be used to identify and isolate receptors that bind zins4 polypeptide. For example, proteins and peptides of the present invention can be immobilized on a column and membrane preparations run over the column (Immobilized Affinity Ligand Techniques, Hermanson et al., eds., Academic Press, San Diego, Calif., 1992, pp.195-202). Proteins and peptides can also be radiolabeled (Methods in Enzvmol., vol. 182, “Guide to Protein Purification”, M.

Deutscher, ed., Acad. Press, San Diego, 1990, 721-37) or photoaffinity labeled (Brunner et al., Ann. Rev. Biochem. 62:483-514, 1993 and Fedan et al., Biochem. Phannacol. 33:1167-80, 1984) and specific cell-surface proteins can be identified.

The polypeptides, antagonists, agonists, nucleic acid and/or antibodies of the present invention may be used in treatment of disorders associated with gonadal development, pregnancy, pubertal changes, menopause, ovarian cancer, fertility, ovarian function, polycystic ovarian syndrome, uterine cancer, endometriosis, libido, mylagia and neuralgia associated with reproductive phenomena, male sexual dysfunction, impotency, prostate cancer, testicular cancer, and dysfunction. The molecules of the present invention may used to modulate or to treat or prevent development of pathological conditions in such diverse tissue as ovary, heart, testis, and kidney. In particular, certain syndromes or diseases may be amenable to such diagnosis, treatment or prevention. Moreover, natural functions, such as embryo implantation or spermatogenesis, may be suppressed or controlled for use in birth control by molecules of the present invention.

Polynucleotides and polypeptides of the present invention will be useful as educational tools in laboratory practicum kits for courses related to genetics and molecular biology, protein chemistry, and antibody production and analysis. Due to its unique polynucleotide and polypeptide sequences, molecules of zins4 can be used as standards or as “unknowns” for testing purposes. For example, zins4 polynucleotides can be used as an aid, such as, for example, to teach a student how to prepare expression constructs for bacterial, viral, or mammalian expression, including fusion constructs, wherein zins4 is the gene to be expressed; for determining the restriction endonuclease cleavage sites of the polynucleotides; determining mRNA and DNA localization of zins4 polynucleotides in tissues (i.e., by northern and Southern blotting as well as polymerase chain reaction); and for identifying related polynucleotides and polypeptides by nucleic acid hybridization.

Zins4 polypeptides can be used as an aid to teach preparation of antibodies; identifying proteins by western blotting; protein purification; determining the weight of produced zins4 polypeptides as a ratio to total protein produced;

identifying peptide cleavage sites; coupling amino and carboxyl terminal tags; amino acid sequence analysis, as well as, but not limited to monitoring biological activities of both the native and tagged protein in vitro and in vivo.

Zins4 polypeptides can also be used to teach analytical skills such as mass spectrometry, circular dichroism to determine conformation, especially of the four alpha helices, x-ray crystallography to determine the three-dimensional structure in atomic detail, nuclear magnetic resonance spectroscopy to reveal the structure of proteins in solution. For example, a kit containing the zins4 can be given to the student to analyze. Since the amino acid sequence would be known by the instructor, the protein can be given to the student as a test to determine the skills or develop the skills of the student, the instructor would then know whether or not the student has correctly analyzed the polypeptide. Since every polypeptide is unique, the educational utility of zins4 would be unique unto itself.

The antibodies which bind specifically to zins4 can be used as a teaching aid to instruct students how to prepare affinity chromatography columns to purify zins4, cloning and sequencing the polynucleotide that encodes an antibody and thus as a practicum for teaching a student how to design humanized antibodies. The zins4 gene, polypeptide, or antibody would then be packaged by reagent companies and sold to educational institutions so that the students gain skill in art of molecular biology. Because each gene and protein is unique, each gene and protein creates unique challenges and learning experiences for students in a lab practicum. Such educational kits containing the zins4 gene, polypeptide, or antibody are considered within the scope of the present invention.

Polynucleotides encoding zins4 polypeptides are useful within gene therapy or gene transfer applications where it is desired to increase or inhibit zins4 activity. If a mammal has a mutated or absent zins4 gene, the zins4 gene can be introduced into the cells of the mammal. In one embodiment, a gene encoding a zins4 polypeptide is introduced in vivo in a viral vector. Such vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Examples of particular vectors include, but are not limited to, a defective herpes simplex virus 1 (HSV1) vector (Kaplitt et al., Molec. Cell. Neurosci. 2:320-30, 1991); an attenuated adenovirus vector, such as the vector described by Stratford-Perricaudet et al., J. Clin. Invest. 90:626-30, 1992; and a defective adeno-associated virus vector (Samulski et al., J. Virol. 61:3096-101, 1987; Samulski et al., J. Virol. 63:3822-8, 1989).

In another embodiment, a zins4 gene can be introduced in a retroviral vector, e.g., as described in Anderson et al., U.S. Pat. No. 5,399,346; Mann et al. Cell 33:153, 1983; Temin et al., U.S. Pat. No. 4,650,764; Temin et al., U.S. Pat. No. 4,980,289; Markowitz et al., J. Virol. 62:1120, 1988; Temin et al., U.S. Pat. No. 5,124,263; International Patent Publication No. WO 95/07358, published March 16, 1995 by Dougherty et al.; and Kuo et al., Blood 82:845, 1993. Alternatively, the vector can be introduced by lipofection in vivo using liposomes. Synthetic cationic lipids can be used to prepare liposomes for in vivo transfection of a gene encoding a marker (Felgner et al., Proc. Natl. Acad. Sci. USA 84:7413-7, 1987; Mackey et al., Proc. Natl. Acad. Sci. USA 85:8027-31, 1988). The use of lipofection to introduce exogenous genes into specific organs in vivo has certain practical advantages. Molecular targeting of liposomes to specific cells represents one area of benefit. More particularly, directing transfection to particular cells represents one area of benefit. For instance, directing transfection to particular cell types would be particularly advantageous in a tissue with cellular heterogeneity, such as the pancreas, liver, kidney, and brain. Lipids may be chemically coupled to other molecules for the purpose of targeting. Targeted peptides (e.g., hormones or neurotransmitters), proteins such as antibodies, or non-peptide molecules can be coupled to liposomes chemically.

It is possible to remove the target cells from the body; to introduce the vector as a naked DNA plasmid; and then to re-implant the transformed cells into the body. Naked DNA vectors for gene therapy or gene transfer can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 267:963-7, 1992; Wu et al., J. Biol. Chem. 263:14621-4, 1988.

Antisense methodology can be used to inhibit zins4 gene transcription, such as to inhibit cell proliferation in vivo. Polynucleotides that are complementary to a segment of a zins4-encoding polynucleotide (e.g., a polynucleotide as set froth in SEQ ID NO: 1) are designed to bind to zins4-encoding mRNA and to inhibit translation of such mRNA. Such antisense polynucleotides are used to inhibit expression of zins4 polypeptide-encoding genes in cell culture or in a subject.

The present invention also provides reagents which will find use in diagnostic applications. For example, the zins4 gene, a probe comprising zins4 DNA or RNA or a subsequence thereof can be used to confim that the zins4 gene is present on human chromosome 19 and can be used as a marker to determine if a mutation has occurred. Zins4 is located in the 19p13.11 region of chromosome 19. Detectable chromosomal aberrations at the zins4 gene locus include, but are not limited to, aneuploidy, gene copy number changes, insertions, deletions, restriction site changes and rearrangements. Such aberrations can be detected using polynucleotides of the present invention by employing molecular genetic techniques, such as restriction fragment length polymorphism (RFLP) analysis, fluorescence in situ hybridization methods, short tandem repeat (STR) analysis employing PCR techniques, and other genetic linkage analysis techniques known in the art (Sambrook et al., ibid.; Ausubel et. al., ibid.). Such probes include, but are not limited to, polynucleotides 74-156, 74-159, 74-162, 163-342, 163-345, 163-348, 163-351, 163-354, 355-426, 1-73, 1-162, 1-342, 74-342, 74-345, 74-348, 74-351, and 74-354.

The precise knowledge of a gene's position can be useful for a number of purposes, including: 1) determining if a sequence is part of an existing contig and obtaining additional surrounding genetic sequences in various forms, such as YACs, BACs or cDNA clones; 2) providing a possible candidate gene for an inheritable disease which shows linkage to the same chromosomal region; and 3) cross-referencing model organisms, such as mouse, which may aid in determining what function a particular gene might have. In general, the diagnostic methods used in genetic linkage analysis, to detect a genetic abnormality or aberration in a patient, are known in the art. Most diagnostic methods comprise the steps of (a) obtaining a genetic sample from a potentially diseased patient, diseased patient or potential non-diseased carrier of a recessive disease allele; (b) producing a first reaction product by incubating the genetic sample with a zins4 polynucleotide probe wherein the polynucleotide will hybridize to complementary polynucleotide sequence, such as in RFLP analysis or by incubating the genetic sample with sense and antisense primers in a PCR reaction under appropriate PCR reaction conditions; (iii) Visualizing the first reaction product by gel electrophoresis and/or other known method such as visualizing the first reaction product with a zins4 polynucleotide probe wherein the polynucleotide will hybridize to the complementary polynucleotide sequence of the first reaction; and (iv) comparing the visualized first reaction product to a second control reaction product of a genetic sample from wild type patient. A difference between the first reaction product and the control reaction product is indicative of a genetic abnormality in the diseased or potentially diseased patient, or the presence of a heterozygous recessive carrier phenotype for a non-diseased patient, or the presence of a genetic defect in a tumor from a diseased patient, or the presence of a genetic abnormality in a fetus or pre-implantation embryo. For example, a difference in restriction fragment pattern, length of PCR products, length of repetitive sequences at the zins4 genetic locus, and the like, are indicative of a genetic abnormality, genetic aberration, or allelic difference in comparison to the normal wild type control. Controls can be from unaffected family members, or unrelated individuals, depending on the test and availability of samples. Genetic samples for use within the present invention include genomic DNA, mRNA, and cDNA isolated form any tissue or other biological sample from a patient, such as but not limited to, blood, saliva, semen, embryonic cells, amniotic fluid, and the like. The polynucleotide probe or primer can be RNA or DNA, and will comprise a portion of SEQ ID NO:1, the complement of SEQ ID NO:1, or an RNA equivalent thereof. Such methods of showing genetic linkage analysis to human disease phenotypes are well known in the art. For reference to PCR based methods in diagnostics see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)).

Aberrations associated with the zins4 locus can be detected using nucleic acid molecules of the present invention by employing standard methods for direct mutation analysis, such as restriction fragment length polymorphism analysis, short tandem repeat analysis employing PCR techniques, amplification-refractory mutation system analysis, single-strand conformation polymorphism detection, RNase cleavage methods, denaturing gradient gel electrophoresis, fluorescence-assisted mismatch analysis, and other genetic analysis techniques known in the art (see, for example, Mathew (ed.), Protocols in Human Molecular Genetics (Humana Press, Inc. 1991), Marian, Chest 108:255 (1995), Coleman and Tsongalis, Molecular Diagnostics (Human Press, Inc. 1996), Elles (ed.) Molecular Diagnosis of Genetic Diseases (Humana Press, Inc. 1996), Landegren (ed.), Laboratory Protocols for Mutation Detection (Oxford University Press 1996), Birren et al. (eds.), Genome Analysis, Vol. 2: Detecting Genes (Cold Spring Harbor Laboratory Press 1998), Dracopoli et al. (eds.), Current Protocols in Human Genetics (John Wiley & Sons 1998), and Richards and Ward, “Molecular Diagnostic Testing,” in Principles of Molecular Medicine, pages 83-88 (Humana Press, Inc. 1998)). Direct analysis of an zins4 gene for a mutation can be performed using a subject's genomic DNA. Methods for amplifying genomic DNA, obtained for example from peripheral blood lymphocytes, are well-known to those of skill in the art (see, for example, Dracopoli et aL (eds.), Current Protocols in Human Genetics, at pages 7.1.6 to 7.1.7 (John Wiley & Sons 1998)).

Sequence tagged sites (STSs) can also be used independently for chromosomal localization. An STS is a DNA sequence that is unique in the human genome and can be used as a reference point for a particular chromosome or region of a chromosome. An STS is defined by a pair of oligonucleotide primers that are used in a polymerase chain reaction to specifically detect this site in the presence of all other genomic sequences. Since STSs are based solely on DNA sequence they can be completely described within an electronic database, for example, Database of Sequence Tagged Sites (dbSTS), GenBank, (National Center for Biological Information, National Institutes of Health, Bethesda, Md. http://www.ncbi.nlm.nih.gov), and can be searched with a gene sequence of interest for the mapping data contained within these short genomic landmark STS sequences. In the present application, polynucleotide primers generating a zins4 sequence mapping to human chromosome 19pl3.11 can be used to generate and STS.

Mice engineered to express the zins4 gene, referred to as “transgenic mice,” and mice that exhibit a complete absence of zins4 gene function, referred to as “knockout mice,” may also be generated (Snouwaert et al., Science 257:1083, 1992; Lowell et al., Nature 366:74042, 1993; Capecchi., Science 244: 1288-92, 1989;

Palmiter et al. Annu Rev Genet. 20: 465-99, 1986). For example, transgenic mice that over-express zins4, either ubiquitously or under a tissue-specific or tissue-restricted promoter can be used to ask whether over-expression causes a phenotype. For example, over-expression of a wild-type zins4 polypeptide, polypeptide fragment or a mutant thereof may alter normal cellular processes, resulting in a phenotype that identifies a tissue in which zins4 expression is functionally relevant and may indicate a therapeutic target for the zins4, its agonists or antagonists. For example, a preferred transgenic mouse to engineer is one that over-expresses the zins4 mature polypeptide (residue 1 (Met) to residue 142 (Cys) of SEQ ID NO:2). Transgenic mice engineered to over-expresses zins4 A chain, B chain or C peptide, can also be used. Moreover, such over-expression may result in a phenotype that shows similarity with human diseases. Similarly, knockout zins4 mice can be used to determine where zins4 is absolutely required in vivo. The phenotype of knockout mice is predictive of the in vivo effects of that a zins4 antagonist, such as those described herein, may have. The human zins4 cDNA can be used to isolate murine zins4 rnRNA, cDNA and genomic DNA, which are subsequently used to generate knockout mice. Relaxin knock out mice have been generated (Zhao et al., Ednocrin. 140:445-52, 1999). Transgenic mice engineered to over-expresses human polypeptides or mouse polypeptides corresponding to the human A chain, B chain or C peptide, can also be used. These mice may be employed to study the zins4 gene and the protein encoded thereby in an in vivo system, and can be used as in vivo models for corresponding human diseases. Moreover, transgenic mice expression of zins4 antisense polynucleotides or ribozymes directed against zins4, described herein, can be used analogously to transgenic mice described above.

A pharmaceutically effective amount of a zins4 polypeptide of the present invention can be formulated with pharmaceutically acceptable carriers for parenteral, oral, nasal, rectal, topical, transdermal administration or the like, according to conventional methods. Formulations may further include one or more diluents, fillers, emulsifiers, preservatives, buffers, excipients, and the like, and may be provided in such forms as liquids, powders, emulsions, suppositories, liposomes, transdermal patches and tablets, for example. Slow or extended-release delivery systems, including any of a number of biopolymers (biological-based systems), systems employing liposomes, and polymeric delivery systems, can also be utilized with the compositions described herein to provide a continuous or long-term source of the zins4 polypeptide or antagonist. Such slow release systems are applicable to formulations, for example, for oral, topical and parenteral use. The term “pharmaceutically acceptable carrier” refers to a carrier medium which does not interfere with the effectiveness of the biological activity of the active ingredients and which is not toxic to the host or patient.

One skilled in the art may formulate the compounds of the present invention in an appropriate manner, and in accordance with accepted practices, such as those disclosed in Remington: The Science and Practice of Pharmacy, Gennaro, ed., Mack Publishing Co., Easton Pa., 19th ed., 1995.

As used herein a “pharmaceutically effective amount” of a zins4 polypeptide, agonists or antagonist is an amount sufficient to induce a desired biological result. The result can be alleviation of the signs, symptoms, or causes of a disease, or any other desired alteration of a biological system. For example, an effective amount of a zins4 polypeptide is that which provides either subjective relief of symptoms or an objectively identifiable improvement as noted by the clinician or other qualified observer. Effective amounts of the zins4 polypeptides can vary widely depending on the disease or symptom to be treated. The amount of the polypeptide to be administered and its concentration in the formulations, depends upon the vehicle selected, route of administration, the potency of the particular polypeptide, the clinical condition of the patient, the side effects and the stability of the compound in the formulation. Thus, the clinician will employ the appropriate preparation containing the appropriate concentration in the formulation, as well as the amount of formulation administered, depending upon clinical experience with the patient in question or with similar patients. Such amounts will depend, in part, on the particular condition to be treated, age, weight, and general health of the patient, and other factors evident to those skilled in the art. Typically a dose will be in the range of 0.1-100 mg/kg of subject. Doses for specific compounds may be determined from in vitro or ex vivo studies in combination with studies on experimental animals. Concentrations of compounds found to be effective in vitro or ex vivo provide guidance for animal studies, wherein doses are calculated to provide similar concentrations at the site of action.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. An isolated polypeptide comprising amino acid residues 26-52 of SEQ ID NO:2.

2. An isolated polypeptide of claim 1, wherein said polypeptide further comprises amino acid residues 119-142 of SEQ ID NO:2.

3. An isolated polypeptide of claim 2, wherein said polypeptide further comprises amino acid residues 55-114 of SEQ ID NO:2.

4. An isolated polypeptide of claim 2, wherein said polypeptide comprises the amino acid residues 26-142 of SEQ ID NO:2.

5. An isolated polypeptide of claim 2, wherein said polypeptide comprises the amino acid residues 1-142 of SEQ ID NO:2.

6. An isolated polypeptide consisting of the amino acid sequence of SEQ ID NO:2.

7. An isolated polypeptide selected from the group consisting of:
a) a polypeptide consisting of the sequence of amino acid residues from residue 26 to residue 52 of SEQ ID NO:2;
b) a polypeptide consisting of the sequence of amino acid residues from residue 26 to residue 53 of SEQ ID NO:2;
c) a polypeptide consisting of the sequence of amino acid residues from residue 26 to residue 54 of SEQ ID NO:2;
d) a polypeptide consisting of the sequence of amino acid residues from residue 55 to residue 114 of SEQ ID NO:2;
e) a polypeptide consisting of the sequence of amino acid residues from residue 55 to residue 115 of SEQ ID NO:2;
f) a polypeptide consisting of the sequence of amino acid residues from residue 55 to residue 116 of SEQ ID NO:2;
g) a polypeptide consisting of the sequence of amino acid residues from residue 55 to residue 117 of SEQ ID NO:2;
h) a polypeptide consisting of the sequence of amino acid residues from residue 55 to residue 118 of SEQ ID NO:2;
i) a polypeptide consisting of the sequence of amino acid residues from residue 119 to residue 142 of SEQ ID NO:2;
j) a polypeptide consisting of the sequence of amino acid residues from residue 26 to residue 114 of SEQ ID NO:2;
k) a polypeptide consisting of the sequence of amino acid residues from residue 26 to residue 118 of SEQ ID NO:2;
l) a polypeptide consisting of the sequence of amino acid residues from residue 55 to residue 142 of SEQ ID NO:2;
m) a polypeptide consisting of the sequence of amino acid residues from residue 1 to residue 25 of SEQ ID NO:2;
n) a polypeptide consisting of the sequence of amino acid residues from residue 1 to residue 52 of SEQ ID NO:2;
o) a polypeptide consisting of the sequence of amino acid residues from residue 26 to residue 54 of SEQ ID NO:2; and
p) a polypeptide consisting of the sequence of amino acid residues from residue 1 to residue 118 of SEQ ID NO:2.

8. An isolated protein comprising:
a B chain comprising amino acid residue 26 to amino acid residue 52 of SEQ ID NO:2; and
an A chain comprising amino acid residue 119 to amino acid residue 142 of SEQ ID NO:2;
wherein the B chain and A chain are joined by inter- and intra-chain disulfide bonds.

9. An isolated polypeptide according to claim 1, further comprising an affinity tag.

10. An isolated polypeptide according to claim 9, wherein said affinity tag is selected from the group consisting of: poly-histidine tract, protein A, glutathione S transferase, Glu-Glu affinity tag, substance P, Flag peptide, streptavidin binding peptide, maltose-binding protein, and an immunoglobulin domain.

11. An isolated polynucleotide molecule that encodes a polypeptide according to claim 1.

12. An isolated polynucleotide of claim 11, wherein said polynucleotide encodes a polypeptide further comprising amino acid residues 119-142 of SEQ ID NO:2.

13. An isolated polynucleotide of claim 12, wherein said polynucleotide encodes a polypeptide further comprising amino acid residues 55-114 of SEQ ID NO:2.

14. An isolated polynucleotide of claim 12, wherein said polynucleotide encodes a polypeptide further comprising the amino acid residues 26-142 of SEQ ID NO:2.

15. An isolated polynucleotide of claim 12, wherein said polynucleotide encodes a polypeptide comprising the amino acid residues 1-142 of SEQ ID NO:2.

16. An isolated polynucleotide encoding a polypeptide comprising the amino acid sequence of SEQ ID NO:2.

17. An isolated polynucleotide molecule comprising the nucleotide sequence of nucleotides 74 to 426 of SEQ ID NO:1.

18. An isolated polynucleotide molecule of SEQ ID NO:1.

19. An isolated polynucleotide selected from the group consisting of:
a) a polynucleotide consisting of nucleotides 74-156 of SEQ ID NO:1;
b) a polynucleotide consisting of nucleotides 74-159 of SEQ IDNO: 1;
c) a polynucleotide consisting of nucleotides 74-162 of SEQ ID NO:1;
d)a polynucleotide consisting of nucleotides 163-342 of SEQ ID NO:1;
e)a polynucleotide consisting of nucleotides 163-345 of SEQ ID NO:1;
f) a polynucleotide consisting of nucleotides 163-348 of SEQ ID NO:1;
g)a polynucleotide consisting of nucleotides 163-351 of SEQ ID NO:1;
h)a polynucleotide consisting of nucleotides 163-354 of SEQ ID NO:1;
i) a polynucleotide consisting of nucleotides 355-426 of SEQ ID NO:1;
j) apolynucleotide consisting of nucleotides 1-73 of SEQ ID NO:1;
k) a polynucleotide consisting of nucleotides 1-162 of SEQ ID NO:1;
l) a polynucleotide consisting of nucleotides 1-342 of SEQ ID NO:1;
m) a polynucleotide consisting of nucleotides 74-342 of SEQ ID NO:1;
n) a polynucleotide consisting of nucleotides 74-345 of SEQ ID NO:1;
o) a polynucleotide consisting of nucleotides 74-348 of SEQ ID NO:1;
p) a polynucleotide consisting of nucleotides 74-351 of SEQ ID NO:1; and
q) a polynucleotide consisting of nucleotides 74-354 of SEQ ID NO:1.

20. An expression vector comprising the following operably linked elements:
a transcription promoter;
a DNA molecule that encodes a polypeptide according to claim 1; and
a transcription terminator.

21. An expression vector according to claim 20 further comprising a secretory signal sequence operably linked to said polypeptide.

22. An expression vector according to claim 20, wherein said polynucleotide encodes a polypeptide covalently linked amino terminally or carboxy terminally to an affinity tag.

23. A cultured cell into which has been introduced an expression vector comprising the following operably linked elements:
a transcription promoter;
a polynucleotide molecule that encodes a polypeptide according to claim 1; and
a transcription terminator, wherein said cultured cell expresses said polypeptide encoded by said polynucleotide segment.

24. A culture cell according to claim 23, wherein said cell further comprises a second expression vector comprising the following operably linked elements:
a transcriptional promoter;
a DNA sequence encoding a prohormone convertase; and
a transcriptional terminator.

25. A cultured cell according to claim 24, wherein said prohormone convertase is selected from the group consisting of prohormone convertase ⅓, prohormone convertase 2, prohormone convertase 4, PACE, PACE4, furin, and kex2.

26. A method of producing a protein comprising:
culturing a cell into which has been introduced an expression vector comprising the following operably linked elements:
a transcription promoter; a polynucleotide molecule that encodes a polypeptide according to claim 1; and a transcription terminator;
whereby said cell expresses said polypeptide encoded by said polynucleotide segment; and
recovering said expressed protein.

27. A method of producing a protein according to claim 26, wherein said cell further comprises a second expression vector comprising the following operably linked elements:
a transcriptional promoter; a DNA sequence encoding a prohormone convertase; and a transcriptional terminator.

28. A method of producing a protein according to claim 27, wherein said prohormone convertase is selected from the group consisting of prohormone convertase 2, prohormone convertase 3, prohormone convertase 4 and furin.

29. An antibody or antibody fragment that specifically binds to a polypeptide according to claim 1.

30. A polypeptide according to claim 1, in combination with a pharmaceutically acceptable vehicle.


SEQUENCE LISTING (12 sequences)

SEQ ID NO: 1 - DNA, 429 bp, Homo sapiens
  CDS (1)...(429)
atggccaggtacatgctgctgctgctcctggcggtatgggtgctgaccggggagctgtggccgggagctgaggcccgggcagcgccttacggggtcaggctttgcggccgagaattcatccgagcagtcatcttcacctgcgggggctcccggtggagacgatcagacatcctggcccacgaggctatgggagataccttcccggatgcagatgctgatgaagacagtctggcaggcgagctggatgaggccatggggtccagcgagtggctggccctgaccaagtcaccccaggccttttacagggggcgacccagctggcaaggaacccctggggttcttcggggcagccgagatgtcctggctggcctttccagcagctgctgcaagtgggggtgtagcaaaagtgaaatcagtagcctttgctag

SEQ ID NO: 2 - protein, 142 aa, Homo sapiens
MARYMLLLLLAVWVLTGELWPGAEARAAPYGVRLCGREFIRAVIFTCGGSRWRRSDILAHEAMGDTFPDADADEDSLAGELDEAMGSSEWLALTKSPQAFYRGRPSWQGTPGVLRGSRDVLAGLSSSCCKWGCSKSEISSLC

SEQ ID NO: 3 - protein, 14 aa, Artificial Sequence
  Cysteine motif
LCGXXXXXXXXXXC

SEQ ID NO: 4 - protein, 15 aa, Artificial Sequence
  Motif
CCXXXCXXXXXXXXC

SEQ ID NO: 5 - protein, 5 aa, Artificial Sequence
  Motif
RXXXR

SEQ ID NO: 6 - DNA, 426 bp, Artificial Sequence
  Degenerate polynucleotide sequence encoding the polypeptide of SEQ ID NO2.
atggcnmgntayatgytnytnytnytnytngcngtntgggtnytnacnggngarytntggccnggngcngargcnmgngcngcnccntayggngtnmgnytntgyggnmgngarttyathmgngcngtnathttyacntgyggnggnwsnmgntggmgnmgnwsngayathytngcncaygargcnatgggngayacnttyccngaygcngaygcngaygargaywsnytngcnggngarytngaygargcnatgggnwsnwsngartggytngcnytnacnaarwsnccncargcnttytaymgnggnmgnccnwsntggcarggnacnccnggngtnytnmgnggnwsnmgngaygtnytngcnggnytnwsnwsnwsntgytgyaartggggntgywsnaarwsngarathwsnwsnytntgy

SEQ ID NO: 7 - DNA, 25 bp, Artificial Sequence
  Oligonucleotide ZC9736
ccatacccctgacccctgttgagat

SEQ ID NO: 8 - DNA, 25 bp, Artificial Sequence
  Oligonucleotide ZC9740
cagaggttccctgatacccacacat

SEQ ID NO: 9 - DNA, 55 bp, Artificial Sequence
  Exon 1 sense oligonucleotide primer
tgaagaaggtctcgaattcgtcgacaccatggccaggtacatgctgctgctgctc

SEQ ID NO: 10 - DNA, 45 bp, Artificial Sequence
  Exon 1 antisense oligonucleotide primer
tgaagaaggtctcactcccatagcctcgtgggccaggatgtctga

SEQ ID NO: 11 - DNA, 41 bp, Artificial Sequence
  Exon 2 sense oligonucleotide primer
tgaagaaggtctcaggagataccttcccggatgcagatgct

SEQ ID NO: 12 - DNA, 52 bp, Artificial Sequence
  Exon 2 antisense oligonucleotide primer
tgaagaaggtctctctagaactctagcaaaggctactgatttcacttttgct